(19) United States
(12) Patent Application Publication
(10) Pub. No.: US 2026/0216082 A1
(43) Pub. Date: Jul. 30, 2026

(54) Controlled-Release Oral Formulations of Lipophilic Drugs

(71) Applicant: BSD SCIENCE LTD., Sevut Rachel (IL)

(72) Inventor: Yochai Yakovson, Sevut Rachel (IL)

(21) Appl. No.: 19/144,334

(22) PCT Filed: Dec. 24, 2023
(86) PCT No.: PCT/IL2023/051300

§ 371 Date: Jun. 27, 2025

Related U.S. Application Data
(60) Provisional application No. 63/435,628, filed on Dec. 28, 2022.

(51) Int. Cl. A61K 9/48; A61K 9/00; A61K 9/06; A61K 9/107; A61K 9/16; A61K 9/70; A61K 31/00; A61K 31/216; A61K 31/427; A61K 36/185; A61K 38/08; A61K 47/26; A61K 47/34; A61K 47/38

(52) U.S. Cl. CPC A61K 9/4808; A61K 9/0053; A61K 9/06; A61K 9/1075; A61K 9/1617; A61K 9/1641; A61K 9/4825; A61K 9/4866; A61K 9/7007; A61K 31/216; A61K 31/427; A61K 31/658; A61K 36/3482; A61K 38/08; A61K 47/26; A61K 47/34; A61K 47/38

(57) ABSTRACT

Oral drug delivery device for controlled delivery of lipophilic drugs, the device comprising film-forming hydrophilic polymer, release-modifying agent, and a lipophilic drug emulsified in an emulsifying agent, wherein the device is integrated into or is formed as an orally administrable dosage unit form, where upon ingestion of the dosage unit form and contact with gastric and/or intestinal medium, the release-modifying agent forms a viscous gel that gradually erodes while travelling through the stomach and the intestinal tract, releasing the emulsified drug into surrounding environment at a controlled rate.

### TECHNOLOGICAL FIELD

Disclosed are orally administered delivery devices and dosage forms thereof for controlled delivery of drugs, and their uses in medicine.

### PRIOR ART

References considered to be relevant as background to the presently disclosed subject matter are listed below:
1. Zanchetta, Beatriz, Marco Vinícius Chaud, and Maria Helena Andrade Santana. “Self-emulsifying drug delivery systems (SEDDS) in pharmaceutical development.” J Adv Chem Eng 5.3 (2015): 1-7.
2. Thakare, Priya, et al. “A review on self-emulsified drug delivery system.” Journal of pharmaceutical and biological evaluations 3.2 (2016): 140-153.)
3. Kalepu, Sandeep, Mohanvarma Manthina, and Veerabhadhraswamy Padavala. “Oral lipid-based drug delivery systems—an overview.” Acta Pharmaceutica Sinica B 3.6 (2013): 361-372.
4. Xiao, Lu, Tao Yi, and Ying Liu. “A new self-microemulsifying mouth dissolving film to improve the oral bioavailability of poorly water-soluble drugs.” Drug Development and Industrial Pharmacy 39.9 (2013): 1284-1290.
5. Izgelov, Dvora, Michael Freidman, and Amnon Hoffman. “Investigation of cannabidiol gastro retentive tablets based on regional absorption of cannabinoids in rats.” European Journal of Pharmaceutics and Biopharmaceutics 152 (2020): 229-235.
6. Punyamurthula, Nagendra S., et al. “Controlled release tablet formulation containing natural Δ9-tetrahydrocannabinol.” Drug development and industrial pharmacy 42.7 (2016): 1158-1164.
7. U.S. Pat. No. 10,004,684B2
8. U.S. Pat. No. 9,265,724B2
9. WO2020014776A1
10. WO2018011798A1
11. WO2009117819A1
12. U.S. Pat. No. 6,399,086B1
13. US20070160677A1
14. IN354423B
15. CN112076177A
16. U.S. Pat. No. 9,358,205B2

### TECHNOLOGICAL BACKGROUND

### Lipophilic Drugs and Lipid-Based Drug Delivery (LBDD) Systems

Poor drug absorption is commonly associated with active pharmaceutical ingredients (APIs) having low aqueous solubility and/or poor intestinal permeability. Amongst other approaches, drug solubilization is improved in the dose form itself and importantly, in the gastrointestinal (GI) environment by employing lipid-based drug delivery (LBDD) systems. The absorption of drug from lipid-based formulation depends on numerous factors, including particle size, degree of emulsification, rate of dispersion and precipitation of drug upon dispersion [1].

### Lipid Digestion and Absorption

Lipid digestion begins in the stomach by gastric lipase/s, secreted continuously. Gastric lipase activity is pH-dependent, being inactive in fasted conditions (gastric pH 1-2), and reaching maximal activity in fed (post-prandial) conditions (gastric pH 4-5.5). The presence of lipids and fatty acids in the stomach triggers a chain of rapid, hormonally prompted events, leading to secretion of biliary and pancreatic substances such as bile salts and phospholipids, which along with pancreatic lipases in the duodenum and the small intestine create the commonly known fed state in the GI (gastrointestinal) tract, whereby the dispersion, emulsification, micellization, and formation of lamellar structures help sustain the drug solubilization in the gut [2, 3].

The process of lipid digestion and absorption is most efficient. As the pre-digested content of the stomach empties into the duodenum, it mixes with bile salts and surfactants, essential for the dispersion of hydrophobic lipid structures within the water-rich environment of the small intestine. Here, the digested lipids components are released at the outer layer of the mixed micelles. Lipases convert the tri- and di-glycerides into monoesters and free fatty acids. As emulsification continues, subsequent reduction in lipid droplet size increases the surface area of lipid droplets, facilitating additional lipolytic action by intestinal lipases. The resulting micelles and lamellar structures trigger a further increase in solubilization capacity.

A lipid pharmaceutical formulation should have the necessary make-up (oil, surfactant and cosurfactant or solvent) to self-emulsify in the gut in the presence of endogenous bile salts and pancreatic secretions. Some pharmaceutical excipients are self-emulsifying and can alone solubilize the API. Otherwise, formulations need to be customized to meet requirements for the API to be absorbable and effective. Low HLB (Hydrophilic-Lipophilic Balance) glycerides (oils) are rapidly lipolyzed in the stomach and then in the duodenum into their respective esters, diesters and free fatty acid components, required for the emulsification and micellization of the drug in the GI environment [2, 3].

### Self-Emulsifying Lipid Formulations (SELF)

Self-emulsification is the property of lipid systems that form emulsion particles upon contact with aqueous media without the need for mechanical or thermal energy. This can happen with multi-component excipients or formulations that consist of three distinct groups of molecules: oils, surfactants, and co-surfactants or solvents at optimal ratios. The right amount of component/s of each group is necessary for the spontaneous formation of emulsion particles upon contact with aqueous media [1].

### Oral Dosage Forms Containing Self-Emulsifying Drug Delivery Systems (SEDDS)

Capsule filling is the simplest and most common technology for the encapsulation of liquid or semisolid self-emulsifying (SE) formulations for oral route delivery. Other solid SE dosage forms have emerged in recent years, such as incorporation of liquid/semisolid SE ingredients into powders/nanoparticles by different solidification techniques (e.g., adsorption to solid carriers, spray drying, melt extrusion, nano-particle technology). However, SEDDS are usually limited to liquid dosage forms because many excipients used in SEDDS are not solids at room temperature [1, 7, 8]. Another example of SEEDS solid formulation is a mouth dispersible or dissolving thin film, from which the drug is absorbed via the oral mucosa [4, 7, 9, 15].

### Controlled Release (CR) Formulations

Controlled release (CR) dosage forms can be designed to release the drug along the GI tract in controlled or sustained profile. Major benefits of these formulations compared to immediate release (IR) formulations are prolonged release, long duration of effect and flat and stable PK profile-leading to less adverse effects.

The major technique for CR formulation involves the use of hydrophilic polymeric matrices, the API being dispersed or dissolved in the matrix. Along the GI tract the matrix is slowly eroded and dissolved and releases the drug in a CR profile [11, 12, 16]. This method may be suitable for hydrophilic drugs. However, a lipophilic drug embedded in a hydrophilic polymeric matrix is not released to the surrounding aqueous environment, and no release occurs.

Another manner to achieve CR of lipophilic drugs may be the use of CR lipids like Precirol® and Compritrol® [6]. A disadvantage of this technique is that in order to achieve efficient absorption, action by gastric lipases and bile salts in the stomach, and thereafter in the duodenum is required. A lipophilic drug which is only and first released in the small intestinal is not capable of becoming emulsified and absorbed.

### GR Formulation

Another manner to achieve CR of lipophilic drugs is by use of GR (Gasrto-Retentive) formulations—with which all of the drug content is released within the stomach, and can be processed by gastric lipases and released bile salts [5, 10]. However, GR formulations are most sensitive to gastric food content and gastric pH effects, and rapid gastric evacuation significantly affects the drug release rate, which may lead to dose dumping. In addition, GR formulations are generally limited to low drug load.

### Delayed Formulations

Delayed immediate release of lipophilic drugs that are embedded in enteric matrix or gel so as to achieve acid protection have been described in various publications. These publications refer to acid-sensitive APIs, embedded in non-soluble gastric matrix which prevents the drug-acid reaction. Once the matrix is evacuated from the stomach, it quickly dissolves and the drug is released in immediate profile.

### SUMMARY OF INVENTION

Disclosed herein is an oral drug delivery device for controlled delivery of a lipophilic drug, the device comprising a pharmaceutically acceptable film-forming hydrophilic polymer, a pharmaceutically acceptable release-modifying agent, and a lipophilic drug emulsified in an emulsifying agent, wherein the device is integrated into or is formed as an orally administrable dosage unit form, and wherein upon ingestion of the dosage unit form and contact with gastric and/or intestinal medium, the release-modifying agent forms a viscous gel that gradually erodes concurrently with passage through the stomach and the intestinal tract, thus releasing the emulsified drug into surrounding environment in the form of micelles at a controlled rate.

Disclosed herein is an oral drug delivery device for controlled delivery of at least one lipophilic drug, the device comprising at least one pharmaceutically acceptable film-forming hydrophilic polymer, at least one pharmaceutically acceptable release-modifying agent, optionally at least one pharmaceutically acceptable plasticizer, and at least one lipophilic drug emulsified in at least one emulsifying agent, wherein the oral drug delivery device is configured for being integrated into or is formed as an orally administrable dosage unit form, and wherein said oral drug delivery device is configured for enabling said at least one release-modifying agent, upon ingestion of said dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles.

In the disclosed an oral drug delivery device, the at least one pharmaceutically acceptable film-forming hydrophilic polymer, the at least one pharmaceutically acceptable release-modifying agent and the optional at least one pharmaceutically acceptable plasticizer form a controlled release (CR) drug-containing polymeric component of the delivery device in which the at least one lipophilic drug emulsified in at least one emulsifying agent is embedded in the polymeric component.

Also disclosed is an oral drug delivery device for controlled delivery of at least one lipophilic drug, where the device comprises a controlled release (CR) drug-containing polymeric component comprising at least one pharmaceutically acceptable film-forming hydrophilic polymeric component, at least one pharmaceutically acceptable release-modifying agent, optionally at least one pharmaceutically acceptable plasticizer, and at least one lipophilic drug emulsified in at least one emulsifying agent embedded in the said polymeric component, wherein the oral drug delivery device is configured for being integrated into or is formed as an orally administrable dosage unit form, wherein said oral drug delivery device is configured for enabling said at least one release-modifying agent, wherein upon ingestion of the said dosage unit form and contact with gastric medium and/or intestinal medium, the device forms a viscous gel that is configured for gradually eroding concurrently with passage through the stomach and the intestinal tract, and for controllably releasing said emulsified drug into surrounding environment in the form of micelles.

In specific embodiments of the disclosed oral drug delivery device, the release-modifying agent is a hydrophilic polymer which when dissolved in water at 2% w/w gives a solution with viscosity of at least 150 mPa*sec.

In specific embodiments of the disclosed oral drug delivery device the release modifying agent is comprised in an amount of at least about 30 mg per said dosage unit form.

In specific embodiments of the disclosed oral drug delivery device the dosage unit form is of total weight of from about 250 to about 2000 mg, preferably from about 300 to about 1200 mg.

In specific embodiments of the disclosed oral drug delivery device the at least one lipophilic drug has log P>2. In disclosed embodiments, the at least one emulsified drug is a pharmaceutically active cannabinoid, or a mixture of at least two pharmaceutically active cannabinoids or a cannabis extract, ritonavir, fenofibrate, diclofenac, flurbiprofen diazepam, vitamin E, piroxicam, lopinavir, clofazimine, or a modified lipophilic drug such as a hydrophobic ion paired peptide drug, pharmaceutically acceptable derivatives and pharmaceutically active metabolites thereof, more specifically a pharmaceutically active cannabinoid, or a mixture of at least two pharmaceutically active cannabinoids, or a cannabis extract.

In specific embodiments, the disclosed oral drug delivery device comprises from about 0.5 to about 500 mg, preferably from about 1 to about 300 mg, more preferably from about 5 to about 300 mg of said lipophilic drug per dosage unit form.

The said dosage unit form can comprise the disclosed drug delivery device integrated into a peroral capsule, preferably a soft gel capsule.

In other embodiments, the disclosed dosage unit form can comprise said CR drug-containing polymeric component integrated into a peroral capsule, such as a hard or soft gel capsule.

A dosage unit form according to the present disclosure, can further comprise an immediate release IR (immediate release) drug-containing component, which IR component comprises an emulsion of the said at least one lipophilic drug in at least one emulsifying agent, the IR drug-containing component optionally further comprising a suitable carrier or diluent, wherein the said IR drug-containing component can be in solid, such as powder, or liquid form. The said capsule can be coated with an immediate release (IR) drug-containing component, which IR component comprises an emulsion of the said at least one lipophilic drug in at least one emulsifying agent, and optionally further comprises a suitable carrier or diluent.

An oral drug delivery device of the present disclosure can comprise from 1 to about 50 CR drug-containing laminated layers, each said CR drug-containing layer comprising the said at least one emulsified lipophilic drug embedded in the said polymeric component, each layer being of pre-determined geometric shape, such as essentially rectangular, square or other polygonal shape or non-polygonal shape, for example elliptical shape, preferably wherein the said layers are each of the same said geometric shape. According to the present disclosure, the thickness of each said drug-containing layer can be from about 20 μm to about 1000 μm, said layers each having identical or different thickness.

The said laminated drug-containing layers of the presently disclosed oral drug delivery device can be rolled together into an essentially cylindrical body. The said drug-containing member of the presently disclosed oral drug delivery can be integrated into a peroral capsule such as a soft gel capsule, and said capsule can optionally further comprise or is coated with an immediate release (IR) drug-containing component which comprises an emulsion of at least one lipophilic drug in at least one emulsifying agent, the IR drug-containing component optionally further comprising a suitable carrier or diluent wherein said IR drug-containing component is in solid or liquid form.

In specific aspects and embodiments of the oral drug delivery device of the present disclosure, the at least one is a pharmaceutically acceptable film-forming hydrophilic polymer, the at least one pharmaceutically acceptable release-modifying agent, the optional at least one pharmaceutically acceptable plasticizer, and the at least one lipophilic drug emulsified in at least one emulsifying agent constitute an essentially solid homogenous CR mixture, configured for being integrated into the said orally administrable dosage unit form.

In some aspects and specific embodiments of the presently disclosed oral drug delivery, the said at least one pharmaceutically acceptable film-forming hydrophilic polymeric component, the said at least one pharmaceutically acceptable release-modifying agent, the said at least one optional pharmaceutically acceptable plasticizer, and the said at least one lipophilic drug emulsified in the said at least one emulsifying agent embedded in therein, constitute an essentially homogenous essentially solid CR mixture, configured for being integrated into said orally administrable dosage unit form. The said CR mixture can be essentially shaped as a cylinder, which may have a diameter of from about 0.2 cm to about 1 cm.

The oral drug delivery device of the present disclosure can be integrated into a peroral capsule, preferably a soft gel capsule, wherein said capsule optionally further comprises and/or is coated with an immediate release (IR) drug-containing component, which comprises an emulsion of said at least one lipophilic drug in at least one emulsifying agent, said IR drug-containing component optionally further comprising a suitable carrier or diluent wherein said IR drug-containing component is in solid or liquid form.

Alternatively, the presently disclosed oral drug delivery device can be configured as a CR orally administrable tablet, the tablet comprising the at least one pharmaceutically acceptable film-forming hydrophilic polymeric, the at least one pharmaceutically acceptable release-modifying agent, optionally the said at least one pharmaceutically acceptable plasticizer, and said at least one lipophilic drug emulsified in at least one emulsifying agent embedded in said constituents. In some such specific embodiments, the tablet comprises a first film-forming polymer and optionally a plasticizer, and the at least one lipophilic drug emulsified in at least one emulsifying agent embedded therein, and also comprises a release modifying agent optionally together with a further film-forming polymer which may be the same or different from said first film-forming polymer, wherein the said tablet optionally further comprises at least one tableting excipient or additive, wherein the excipient or additive is any one of binder, diluent, filler, lubricant and glidant, and any mixture of at least two thereof. The said tablet can optionally further comprise a further amount of at least one additional pharmaceutically acceptable release-modifying polymer in solid form, preferably in ground or powder form, which additional release modifying is the same or different from said at least one, respectively, additional, pharmaceutically acceptable release-modifying agent.

The disclosed tablet-formed oral drug delivery device may be optionally coated with an IR drug-containing component, which component comprises an emulsion of said at least one lipophilic drug in at least one emulsifying agent, said IR drug-containing component optionally further comprising a suitable carrier or diluent wherein said IR drug-containing component is in solid or liquid form.

Further, in all embodiments of the tablet-formed oral drug delivery device disclosed herein, the tablet can be contained in a peroral capsule, such as a soft gel capsule, the capsule optionally further comprising an immediate release (IR) drug-containing component comprising an emulsion of at least one lipophilic drug in at least one emulsifying agent, the IR drug-containing component optionally further comprising a suitable carrier or diluent wherein said IR drug-containing component is in solid or liquid form.

In some specific aspects and embodiments, the oral drug delivery device disclosed herein comprises the at least one pharmaceutically acceptable film-forming hydrophilic polymer and the optional at least one pharmaceutically acceptable plasticizer form a drug-containing polymeric component in which component the at least one lipophilic emulsified drug is embedded; and at least one pharmaceutically acceptable release-modifying agent and optionally at least one pharmaceutically acceptable plasticizer forming a CR polymeric component which may optionally further comprise at least one pharmaceutically acceptable film-forming hydrophilic polymer; where the hydrophilic film-forming polymer and the optional plasticizer in the drug-containing polymeric component and in the CR polymeric component may be the same or different.

In further specific aspects and embodiments, disclosed herein is an oral drug delivery device for controlled delivery of at least one lipophilic drug, the device comprising a drug-containing polymeric component comprising at least one pharmaceutically acceptable film-forming hydrophilic polymeric component, optionally at least one pharmaceutically acceptable plasticizer, in which drug-containing polymeric component the emulsified lipophilic drug is embedded; a controlled release (CR) polymeric component comprising at least one pharmaceutically acceptable film-forming hydrophilic polymer, at least one pharmaceutically acceptable release-modifying agent, and optionally at least one pharmaceutically acceptable plasticizer, wherein said hydrophilic film-forming polymer and said optional plasticizer in said drug-containing polymeric component and in said CR polymeric component may be the same or different; the device being configured for being integrated into an orally administrable dosage unit form, and the device being configured for enabling the at least one release-modifying agent, upon ingestion of said dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles. The drug-containing polymeric component in devices of these aspects and embodiments can comprise from 1 to about 50 drug-containing layers, the drug-containing layers being laminated, each drug-containing layer being of pre-determined geometric shape, such as essentially rectangular, square or other polygonal shape, or non-polygonal such as for example of elliptical shape, more specifically where the layers are of the same said geometric shape. The thickness of each drug-containing layer can be from about 15 μm to about 900 μm, the same or different for each of the layers. In these specific embodiments, the drug-containing polymeric component and the CR polymeric component can also be laminated together, and these laminated drug-containing layer and CR polymeric component can be rolled together to form an essentially cylindrical body. Devices of these embodiments can be integrated into a peroral capsule, such as a soft gel capsule, the capsule optionally further comprising and/or is coated with an immediate release (IR) drug-containing component, which comprises an emulsion of at least one lipophilic drug in at least one emulsifying agent, the drug being the same or different from the drug contained in said CR drug-containing layer, where the IR drug-containing component optionally further comprises a suitable carrier or diluent, and wherein the IR drug-containing component is in solid or liquid form.

Further, disclosed herein is an oral drug delivery device for controlled delivery of at least one lipophilic drug, the device comprising at least one pharmaceutically acceptable film-forming hydrophilic polymer, at least one pharmaceutically acceptable release-modifying agent, optionally at least one pharmaceutically acceptable plasticizer, and at least one lipophilic drug emulsified in at least one emulsifying agent, wherein the oral drug delivery device is configured for being integrated into or is formed as an orally administrable dosage unit form, the oral drug delivery device being configured for enabling said at least one release-modifying agent, upon ingestion of the dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles, wherein said release-modifying agent is a hydrophilic polymer which when dissolved in water at 2% w/w gives a solution with viscosity of at least 150 mPa*sec, wherein said release modifying agent is comprised in an amount of at least about 30 mg per said dosage unit form and wherein said at least one emulsifying agent is a lipid excipient having HLB (Hydrophilic Lipophilic Balance) greater than 6, optionally wherein said emulsifying agent is a mixture of lipid excipients of which at least 50% have HLB greater than 6, and remaining lipid excipients are any one of oils, fatty acids, glycerides, polyoxyglycerides, polyglyceryl and polyalcohol esters or water insoluble surfactants, or any mixture of at least two thereof, wherein each said remaining lipid excipients has HLB lower than, equal to or higher than 6.

Still further, disclosed herein is an oral drug delivery device for controlled delivery of at least one lipophilic drug, the device comprising a CR drug-containing polymeric component which comprises at least one pharmaceutically acceptable film-forming hydrophilic polymeric component, at least one pharmaceutically acceptable release-modifying agent, optionally at least one pharmaceutically acceptable plasticizer, and at least one lipophilic drug emulsified in at least one emulsifying agent embedded in the polymeric component, the device being configured for being integrated into or is formed as an orally administrable dosage unit form, and the device being configured for enabling the at least one release-modifying agent, upon ingestion of said dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles, wherein said release-modifying agent is a hydrophilic polymer which when dissolved in water at 2% w/w gives a solution with viscosity of at least 150 mPa*sec, wherein said release modifying agent is comprised in an amount of at least about 30 mg per said dosage unit form and wherein said at least one emulsifying agent is a lipid excipient having HLB (Hydrophilic Lipophilic Balance) greater than 6, optionally wherein said emulsifying agent is a mixture of lipid excipients of which at least 50% have HLB greater than 6, and remaining lipid excipients are any one of oils, fatty acids, glycerides, polyoxyglycerides, polyglyceryl and polyalcohol esters or water insoluble surfactants, or any mixture of at least two thereof, wherein each said remaining lipid excipients has HLB lower than, equal to or higher than 6.

Still further, disclosed herein is an oral drug delivery device for controlled delivery of at least one lipophilic drug, the device comprising a drug-containing polymeric component comprising at least one pharmaceutically acceptable film-forming hydrophilic polymeric component, optionally at least one pharmaceutically acceptable plasticizer, and at least one lipophilic drug emulsified in at least one emulsifying agent, wherein said emulsified drug is embedded in said polymeric component, a controlled release (CR) polymeric component comprising at least one pharmaceutically acceptable film-forming hydrophilic polymer, at least one pharmaceutically acceptable release-modifying agent, and optionally at least one pharmaceutically acceptable plasticizer, wherein the hydrophilic film-forming polymer and said optional plasticizer in said drug-containing polymeric component and in said CR polymeric component may be the same or different; and wherein the oral drug delivery device is configured for being integrated into an orally administrable dosage unit form, the drug delivery device being configured for enabling said at least one release-modifying agent, upon ingestion of the dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles, wherein said release-modifying agent is a hydrophilic polymer which when dissolved in water at 2% w/w gives a solution with viscosity of at least 150 mPa*sec, wherein the release modifying agent is comprised in an amount of at least about 30 mg per said dosage unit form and wherein said at least one emulsifying agent is a lipid excipient having HLB (Hydrophilic Lipophilic Balance) greater than 6, optionally wherein said emulsifying agent is a mixture of lipid excipients of which at least 50% have HLB greater than 6, and remaining lipid excipients are any one of oils, fatty acids, glycerides, polyoxyglycerides, polyglyceryl and polyalcohol esters or water insoluble surfactants, or any mixture of at least two thereof, wherein each said remaining lipid excipients has HLB lower than, equal to or higher than 6.

Also in these specific embodiments of the disclosed oral drug delivery device the dosage unit form can be of total weight of from about 250 to about 2000 mg, preferably from about 300 to about 1200 mg. The at least one emulsified lipophilic drug can be comprised at from about 1 to about 65% w/w, specifically at from about 5 to about 65% w/w, more specifically at from about 10 to about 65% w/w.

In all aspects and embodiments of the presently disclosed oral drug delivery device, the pharmaceutically active cannabinoid can be any one of Δ-9-tetrahydrocannabinol (Δ9-THC, THC), iso-tetrahydrocannabimol (iso-THC), cannabinol (CBN), cannabidiol (CBD) cannabigerol (CBG), cannabichromene (CBC), cannabielsoin (CBE), cannabicyclol (CBL), cannabicitran (CBT), cannabivarin (CBV), tetrahydro-cannabivarin (THCV), cannabidivarin (CBDV), and many others such as tetrahydrocannabidiol (THCBD), tetrahydrocannabigerol (THCBG), tetrahydrocannabichromene (THCBC), tetrahydrocannabidivarol (THCBDV), cannabichromevarin (CBCV), cannabigerovarin (CBGV) and cannabigerol conomethyl ether (CBGM) and pharmaceutically acceptable derivatives and pharmaceutically active metabolites thereof.

In all aspects and embodiments of the presently disclosed oral drug delivery device, the pharmaceutically active drug can be ritonavir, fenofibrate, diclofenac, flurbiprofen diazepam, vitamin E, piroxicam, lopinavir, clofazimine, or a modified lipophilic drug such as a hydrophobic ion paired peptide drug, pharmaceutically acceptable derivatives and pharmaceutically active metabolites thereof.

In all aspects and embodiments of the presently disclosed oral drug delivery device, the release-modifying agent can be any one of methylcellulose (such as A4M), hydroxypropyl methylcellulose (such as K250, K750,K1500, K4M, E4M,E10M, K15M, K100M or K200M), hydroxypropyl cellulose, (such as Klucel GF, Klucel MF or Klucel HF), hydroxyethyl cellulose (such as HEC G, HEC M, HEC HX, HEC HHX), polyethylene oxide, carboxymethyl cellulose such as (CMC 7MF, CMC 7H3F or CMC 7HF), gelatin, a gum and a protein, and any mixture of at least two thereof.

In all aspects and embodiments of the presently disclosed oral drug delivery device, the emulsifying agent can be a lipid excipient having HLB (Hydrophilic Lipophilic Balance) greater than 6. Such lipid excipient can be any one of Gelucire 50/13 (stearoyl polyoxyl-32 glycerides), Gelucire 44/14 (lauroyl polyoxyl-32 glycerides), Gelucire 48/16 (polyoxyl-32 stearate (type I) NF), Labrafil 2125 (linoleoyl polyoxyl-6 glycerides), Labrafil 1944 (oleoyl polyoxyl-6 glycerides), Labrasol (caprylocaproyl polyoxyl-8 glycerides), Kolliphor RH40 (polyoxyl 40 hydrogenated castor oil), Kolliphor EL (polyoxyl-35 castor oil), Kolliphor HS 15 (macrogol-15-hydroxystearate), Kolliphor P 407/124/188 (poloxamer 407/124/188) and Kolliphor PS 80/60/20 (polysorbate 80/60/20), and any mixture of at least two thereof. The remaining lipid excipients can be any one of oils, fatty acids, glycerides, polyoxyglycerides, polyglyceryl and polyalcohol esters or water insoluble surfactants, or any mixture of at least two thereof, wherein each said remaining lipid excipients has HLB lower than, equal to or higher than 6.

In all aspects and embodiments of the presently disclosed oral drug delivery device, the weight ratio lipophilic drug:emulsifying agent can be from about 2:1 to about 1:100, preferably from about 1:1 to about 1:20.

In all aspects and embodiments of the presently disclosed oral drug delivery device, the hydrophilic film-forming polymer can be any one of povidone, copovidone, polyvinyl alcohol, hydrophilic polyacrylamide derivatives, proteins, gelatin, hydroxypropyl cellulose, polyethylene oxide, amino-methacrylate copolymer NF, hydroxypropyl methylcellulose, hydroxyethyl cellulose, carboxymethyl cellulose, sodium carboxymethylcellulose, methylcellulose, or polyvinyl alcohol-polyethylene glycol graft copolymer and any combination of at least two thereof.

In all aspects and embodiments of the presently disclosed oral drug delivery device, the film-forming polymer and the release-modifying agent can together constitute from about 25 to about 85% w/w, specifically from about 25 to 70% w/w, more specifically from about 30 to about 70% w/w of said device.

In all aspects and embodiments of the present disclosure, the oral drug delivery device may optionally comprise a plasticizer at up to 20% w/w, specifically at up to 15% w/w, more specifically at from about 5% to about 10% w/w.

In further aspects and embodiments, disclosed is an orally administrable pharmaceutical dosage unit form comprising any of the above drug delivery devices that is configured for being integrated into a pharmaceutical dosage unit form, specifically integrated with or into a peroral capsule, specifically a soft gel capsule said capsule. In further aspects and embodiments, the drug delivery device disclosed herein is formed as an orally administrable pharmaceutical dosage unit, specifically formed as a tablet. Also tablets according to the present disclosure can be integrated with or into a peroral capsule, specifically a soft gel capsule said capsule. In orally administrable dosage unit forms in the form of peroral capsule, the capsule may comprise an immediate release IR drug-containing component, which IR component comprises an emulsion of said at least one lipophilic drug in at least one emulsifying agent, the drug in the IR component being identical to or different from the drug contained in said drug delivery device, said IR drug-containing component optionally further comprising a suitable carrier or diluent wherein said IR drug-containing component is in solid, such as powder, or liquid form. The drug may be distributed between the CR drug delivery device and the IR drug-containing component at a pre-determined ratio. Tablets can be coated with IR component comprising the same or other drug as in the CR component. In specific orally administrable pharmaceutical dosage unit forms the active ingredient may be at least one pharmaceutically active cannabinoid or mixture of at least two pharmaceutically active cannabinoids or cannabis extract.

In yet a further aspect of the present disclosure, the disclosed drug delivery devices and the orally administrable pharmaceutical dosage unit forms thereof in which the drug is a cannabinoid or a mixture of cannabinoids or cannabis extract, can be used in methods for any one of treating, alleviating and preventing worsening of a disease, disorder or condition responsive to cannabinoid therapy in a subject in need, said method comprising orally administering to the subject the drug delivery device or pharmaceutical dosage unit form.

The disease, disorder or condition responsive to cannabinoid therapy may be any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia or any other condition responsive to cannabinoid therapy. Administration may be once or twice daily or three times a day. Administration may be chronic.

In yet a further aspect of the present disclosure, in the disclosed drug delivery devices and the orally administrable pharmaceutical dosage unit forms thereof the pharmaceutically active drug can be ritonavir, fenofibrate, diclofenac, flurbiprofen diazepam, vitamin E, piroxicam, lopinavir, clofazimine, or a modified lipophilic drug such as a hydrophobic ion paired peptide drug, pharmaceutically acceptable derivatives and pharmaceutically active metabolites thereof.

Further disclosed are methods for any one of treating, alleviating and preventing worsening of a disease, disorder or condition responsive to cannabinoid therapy in a subject in need, the method comprising orally administering to the subject an oral drug delivery device or a pharmaceutical dosage unit form thereof as disclosed herein in which the drug is a cannabinoid or a mixture of cannabinoids or cannabis extract. Respectively, disclosed are methods for any one of treating, alleviating and preventing worsening of a disease, disorder or condition responsive to ritonavir, fenofibrate, diclofenac, flurbiprofen diazepam, vitamin E, piroxicam, lopinavir, clofazimine, or a modified lipophilic drug such as a hydrophobic ion paired peptide drug, pharmaceutically acceptable derivatives and pharmaceutically active metabolites thereof.

Also disclosed is a method for providing a subject in need thereof with stable therapeutically effective plasma level of at least one cannabinoid or mixture of at least two cannabinoids and/or active metabolites thereof over a prolonged period of time, the method comprising orally administering to the subject an oral drug delivery device or pharmaceutical dosage unit form as disclosed herein in which the drug is a cannabinoid or a mixture of cannabinoids or cannabis extract.

In all the herein disclosed methods of treatment, administration may be once or twice daily or three times a day, or administration may be chronic. Administration may be under fed or fasted conditions.

### BRIEF DESCRIPTION OF THE DRAWINGS

In order to better understand the subject matter that is disclosed herein and to exemplify how it can be carried out in practice, embodiments will now be described, by way of non-limiting example only, with reference to the accompanying drawings, in which:

FIG. 1 CBD release from Formulation 1 of Example 1 in SGF and SIF.

FIG. 2 CBD release from Formulation 2 of Example 2 in SGF and SIF.

FIG. 3 CBD release from Formulation 3 of Example 3 in SGF and SIF.

FIG. 4 CBD release from Formulation 4 of Example 4 in SGF.

FIG. 5 CBD release from Formulation 5 of Example 5 in SGF.

FIG. 5A SEM images of hot melt extrudate prepared in Example 5.

### DETAILED DESCRIPTION OF EMBODIMENTS

### General Description

The present disclosure relates to oral drug delivery devices and dosage forms thereof for controlled delivery of lipophilic drugs and poorly water-soluble drugs, in which devices/dosage forms the drug is contained in an already emulsified form, specifically as a drug emulsion in suitable emulsifying agents, specifically various lipids, fats and/or surfactants as described herein. The presently disclosed device/dosage form provide for improved and prolonged controlled release of the emulsified drug, in the form of micelles, and provide for improved absorption of the drug by a treated subject.

In all aspects and embodiments, the orally administered drug delivery device for controlled release (CR) of at least one emulsified lipophilic drug comprises at least one pharmaceutically acceptable film-forming hydrophilic polymer, at least one pharmaceutically acceptable release-modifying agent, optionally at least one pharmaceutically acceptable plasticizer, and at least one lipophilic drug emulsified in at least one emulsifying agent, wherein the oral drug delivery device is configured for being integrated into or is formed as an orally administrable dosage unit form, and wherein said oral drug delivery device is configured for enabling said at least one release-modifying agent, upon ingestion of said dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles.

Following oral ingestion and upon contact of the delivery device with gastric environment, the delivery device becomes viscous, forming a gradually erodible viscous device or viscous body (which may be used herein interchangingly) which comprises the emulsified drug, the viscous body/device providing for controlled release of the emulsified drug comprised therein, in the form of micelles, over a prolong period of time, starting in the stomach and continuing after the viscous body/device is evacuated from the stomach into the duodenum and then continues its travel along the intestines until it is essentially completely eroded/biodegraded. Thus, in all aspects and embodiments of the present disclosure the drug delivery device and/or dosage forms thereof provides for release and absorption of the emulsified drug over a period of at least about 3 hours and up to about 24 hours, providing stable therapeutically effective and reliable plasma levels for prolonged periods of time. Treatment with the disclosed controlled release oral delivery devices and dosage forms thereof provides for release of the drug soon after administration and along prolonged residence time of the delivery device in the GI tract of the patient, improving both magnitude and duration of the drug pharmacodynamic effects, maximizing therapeutic effects and minimizing any negative side effects. The disclosed oral drug delivery devices and dosage forms enable administration of predetermined therapeutic doses of the drug and reduction of the number of daily administrations, leading to improved patient's compliance.

Specific lipophilic drugs are cannabinoids, as described in more detail below, which are useful in the treatment of various cannabinoid-responsive conditions.

In order to achieve absorption of lipophilic drugs in the body, as mentioned above, such drugs need to be processed and digested in the stomach by gastric lipase/s. leading to secretion of biliary and pancreatic substances such as bile salts and phospholipids, which along with pancreatic lipases in the duodenum and the small intestine lead to the commonly known “fed state” in the GI tract, whereby the dispersion, emulsification, micellization, and formation of lamellar structures help sustain the drug solubilization in the gut. As the pre-digested content of the stomach empties into the duodenum, it mixes with bile salts and surfactants, essential for the dispersion of hydrophobic lipid structures within the water-rich environment of the small intestine. Here, the digested lipids components are released at the outer layer of the mixed micelles. Lipases convert the tri- and di-glycerides into monoesters and free fatty acids. As emulsification continues, subsequent reduction in lipid droplet size increases the surface area of lipid droplets, facilitating additional lipolytic action by intestinal lipases. The resulting micelles and lamellar structures trigger a further increase in solubilization capacity, emulsion and micelles.

Due to the fact that the drug in the presently disclosed delivery device is in form of emulsified drug, it is released to the surrounding medium as drug micelles. The drug micelles can be readily absorbed along all the GI tract, starting in the stomach and continuing into the duodenum and then along the intestines, with no need for pre-digestive processing by gastric lipase/s, bile salts and phospholipids. According to this phenomenon efficient controlled release can be achieved leading to prolonged and stable pharmacokinetic (PK) profile of the lipophilic drug. Thus, the oral drug delivery devices of the present disclosure, and all disclosed dosages unit forms thereof, are not retained in the stomach, rather they travel along the GI tract until they are essentially completely eroded, gradually releasing the active drug contained therein in a controlled manner. Thus, in all aspects and embodiments of the disclosure, the drug delivery device and dosage forms thereof are not gastroretentive.

As shown in Example 11 below, in the absence of a suitable CR component as described herein and presented, for example in Examples 1-5, the resulting delivery device (comprising active ingredients THC and CBD) was completely dissolved within 30-60 minutes.

In some aspects and embodiments of the present disclosure, the oral drug delivery devices and dosages unit forms thereof, the active pharmaceutical ingredient (drug) is mixed with the lipid emulsifying agent at ratios of 2:1 to 1:100 drug: emulsifying agent.

The oral drug delivery device/s of the present disclosure, and all disclosed dosages unit forms thereof comprise constituents which provide for the absorption of the drug in the described desired from, i.e. controlled release of readily absorbable drug micelles upon ingestion, starting in the stomach, and continuing upon emptying to the duodenum and intestines. Specifically, these constituents are that the drug is contained in emulsified form and that the delivery device/dosage form comprises a release adjusting agent that is a hydrophilic polymer which when dissolved in water at 2% w/w gives a solution with viscosity of at least 150 mPa*sec, which is preferably comprised in a dosage unit form in an amount of at least about 30 mg per said dosage unit form, wherein the dosage form is of a total weight of from about 250 to about 2000 mg, and the emulsifying agent is a lipid excipient having HLB (Hydrophilic Lipophilic Balance) greater than 6, or is a mixture of lipid excipients of which at least 50% have HLB greater than 6. These oral drug delivery device and dosage forms thereof provide for prolonged release and efficient absorption of the active drug.

In some specific aspects and embodiments of the delivery device of the present disclosure, the said at least one pharmaceutically acceptable film-forming hydrophilic polymer, said at least one pharmaceutically acceptable release-modifying agent, said optional at least one pharmaceutically acceptable plasticizer are configured to form a controlled release drug-containing polymeric component of the delivery device, and the at least one lipophilic drug emulsified in at least one emulsifying agent (also referred to herein a the drug emulsion) is embedded in said polymeric component.

In some specific aspects and embodiments of the delivery device of the present disclosure, the dosage unit form comprises the said drug delivery device integrated into a peroral capsule, such as a soft gel capsule. In other specific aspects and embodiments of present disclosure, the delivery device is formed as a dosage unit form, for example a tablet.

In one aspect, disclosed herein is an oral drug delivery device for controlled delivery of at least one lipophilic drug, the device comprising a CR drug-containing polymeric component comprising at least one pharmaceutically acceptable film-forming hydrophilic polymeric component, at least one pharmaceutically acceptable release-modifying agent, optionally at least one pharmaceutically acceptable plasticizer, and at least one lipophilic drug emulsified in at least one emulsifying agent embedded in said polymeric component, wherein the oral drug delivery device is configured for being integrated into or is formed as an orally administrable dosage unit form, and wherein said oral drug delivery device is configured for enabling said at least one release-modifying agent, upon ingestion of said dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles.

In specific embodiments of the said aspect of the present disclosure, the drug delivery device comprises a single or multiple laminated CR drug-containing layers, for example up to 6, 8, 10, 12, 15, 20 and up to 50 drug-containing layers. Each CR drug-containing layer polymeric components comprises a hydrophilic film-forming polymer, a release modifying agent and optionally a plasticizer, and at least one lipophilic drug emulsified in at least one emulsifying agent. The CR drug-containing layers are of pre-determined geometric shape for example, but not limited to, essentially rectangular, square or other polygonal shape or non-polygonal shape, such as for example elliptical shape, where said layers each may each be of the same said geometric shape, and also of different geometric shapes. The CR drug-containing layers may each be of an identical or different thickness of from about 20 μm to about 1000 μm. In some specific embodiments, said CR drug-containing layer or laminated CR drug-containing layers are rolled together to form an essentially cylindrical body, or they may be shaped together to a different form or shape. The resulting drug delivery device may be integrated with an oral capsule, for example a soft gel capsule. Specifically, the resulting drug delivery device may be inserted into the oral capsule. Exemplary devices are described in Examples 1-3 below.

In other specific embodiments of said aspect of the present disclosure, the said drug delivery device is configured as a tablet, such as a pharmaceutical tablet, or pill or caplet or the like. An exemplary device is described in Example 12 below. Such tablet comprises at least one pharmaceutically acceptable film-forming hydrophilic polymeric, at least one pharmaceutically acceptable release-modifying agent, optionally at least one pharmaceutically acceptable plasticizer and at least one lipophilic drug emulsified in at least one emulsifying agent, and optionally further comprises at least one tableting excipient or additive, which may be any one of binder, diluent, filler, lubricant and glidant, and any mixture of at least two thereof. A specific tablet may comprise said drug embedded in the hydrophilic film-forming polymer, a first release modifying agent and the optional plasticizer, and optionally at least one tableting excipient or additive. Such tablet may further comprise an amount of a further (second) release modifying agent, which may be the same or different form the first modifying agent.

In another specific embodiment, a tablet according to the present disclosure may comprise said emulsified lipophilic drug embedded in the hydrophilic film-forming polymer and the optional plasticizer, and further comprise the release modifying agent mixed therewith, and optionally the at least one tableting excipient or additive. Generally, the various components or the tablet/s are ground, mixed and then pressed into a tablet. An exemplary device is described in Example 13 below.

Tablets in accordance with the present disclosure may be inserted into peroral capsules.

In the above aspects and embodiments of the present disclosure, wherein the drug delivery device is integrated with or into a peroral capsule, said capsule may further optionally contain and/or be coated with an immediate release (IR) drug-containing component, which comprises an emulsion of at least one lipophilic drug in at least one emulsifying agent, the IR drug-containing component optionally further comprising a suitable carrier or diluent wherein said IR drug-containing component is in solid, for example powder, or liquid form. Exemplary devices are described in Examples 1-3 below.

In embodiments of the presently disclosed delivery device in tablet form, the tablet may optionally be coated with a similar IR drug-containing component which comprises an emulsion of at least one lipophilic drug in at least one emulsifying agent, the IR drug-containing component optionally further comprising a suitable carrier or diluent wherein said IR drug-containing component is in solid, for example powder, or liquid form.

In all aspects and embodiments of the present disclosure the drug comprised in the IR drug-containing component/s may be the same or different from the drug comprised in the CR drug-containing layer/s or component/s.

In other specific embodiments of the disclosed drug-delivery device and dosage forms thereof, the at least one lipophilic drug emulsified in at least one emulsifying agent is embedded in the at least one pharmaceutically acceptable film-forming hydrophilic polymeric component, the at least one pharmaceutically acceptable release-modifying agent, and the optional at least one optional pharmaceutically acceptable plasticizer, the resulting drug-containing polymeric mixture constituting an essentially homogenous essentially solid CR mixture, configured for being integrated into said orally administrable dosage unit form. This device can be produced using hot melt extrusion process as described in more detail below and exemplified in Example 5.

The said CR mixture may be shaped into suitable form, for example as an essentially cylindrical body, for example of a diameter of from about 0.2 cm to about 1 cm.

In a further embodiment, disclosed herein is an oral drug delivery device in which the at least one pharmaceutically acceptable film-forming hydrophilic polymer and the optional at least one pharmaceutically acceptable plasticizer form a drug-containing polymeric component, and the at least one lipophilic drug emulsified in at least one emulsifying agent said emulsified drug is embedded in this polymeric component, and the at least one pharmaceutically acceptable release-modifying agent and optionally at least one pharmaceutically acceptable plasticizer form a CR polymeric component, which CR component may optionally further comprise at least one pharmaceutically acceptable film-forming hydrophilic polymer. In this embodiment, the hydrophilic film-forming polymer and the optional plasticizer in the drug-containing polymeric component and in the CR polymeric component may be the same or different.

Also in this further embodiment, the drug delivery device essentially comprising a CR polymeric component and a drug-containing component, may be integrated into a peroral capsule, such as a soft gel capsule, which capsule may further optionally contain and/or be coated with an immediate release (IR) drug-containing component, which comprises an emulsion of at least one lipophilic drug in at least one emulsifying agent, the IR drug-containing component optionally further comprising a suitable carrier or diluent wherein said IR drug-containing component is in solid, for example powder, or liquid form.

In another aspect, the present disclosure relates to oral drug delivery device for controlled delivery of at least one lipophilic drug, essentially comprising two distinct polymeric components, a drug-containing component and a CR component. An exemplary device is described in Example 4. Briefly, the device according to this embodiment comprises (a) a drug-containing polymeric component comprising at least one pharmaceutically acceptable film-forming hydrophilic polymeric component, optionally at least one pharmaceutically acceptable plasticizer, and at least one lipophilic drug emulsified in at least one emulsifying agent, wherein the emulsified drug is embedded in the polymeric component; (b) a controlled release (CR) polymeric component comprising at least one pharmaceutically acceptable film-forming hydrophilic polymer, at least one pharmaceutically acceptable release-modifying agent, and optionally at least one pharmaceutically acceptable plasticizer, wherein the hydrophilic film-forming polymer and the optional plasticizer in the drug-containing polymeric component and in the CR polymeric component may be the same or different; and wherein the oral drug delivery device is configured for being integrated into an orally administrable dosage unit form, and wherein the oral drug delivery device is configured for enabling the at least one release-modifying agent, upon ingestion of said dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles.

In embodiments of this other aspect of the invention, the drug-containing polymeric component may comprise from 1 to about 50 drug-containing layers, such 1, 2, 3, 4, 5, 6, up to 10, up to 12, up to 15, up to 20, 30, 40 and up to 50 layers, each layer comprising the at least one emulsified lipophilic drug embedded in the hydrophilic polymeric component and optionally said plasticizer, the drug-containing layers being laminated, each said drug-containing layer being of pre-determined geometric shape, preferably of essentially rectangular, square or other polygonal shape or non-polygonal shape, such as for example elliptical shape, preferably wherein said layers each is of the same said geometric shape. The thickness of each of the drug-containing layers is from about 15 μm to about 900 μm, and may be the same or different for each layer.

In specific embodiments of this other aspect of the present disclosure, the drug-containing polymeric component and the CR polymeric component may be laminated together. The laminated drug-containing polymeric component and CR polymeric component may be rolled together to form an essentially cylindrical body, or formed as a body of another configuration, for example an elongated block.

Also in this other aspect of the invention, the drug delivery device may be integrated into a peroral capsule, for example a soft gel capsule, the capsule optionally further comprising and/or is coated with an immediate release (IR) drug-containing component, which comprises an emulsion of said at least one lipophilic drug in at least one emulsifying agent, said IR drug-containing component optionally further comprising a suitable carrier or diluent wherein said IR drug-containing component is in solid or liquid form.

In all aspects and embodiments of the present disclosure, the release-modifying agent is a hydrophilic polymer which when dissolved in water at 2% w/w gives a solution with viscosity of at least 150 mPa*sec. Exemplary release-modifying agents are, but not limited to hydroxypropyl cellulose (such as Klucel GF (viscosity of 150-400 mPa*sec), Klucel MF (viscosity of 4000-6500 mPa*sec) or Klucel HF (viscosity of 3000-6000 mPa*sec)), hydroxypropyl methylcellulose (HPMC) (such as HPMC K250 (viscosity of 200-300 mPa*sec), K750 (viscosity of 600-900 mPa*sec), K1500 (viscosity of 1200-1800 mPa*sec), K4M (viscosity of 2700-5040 mPa*sec), E4M (viscosity of 2700-5040 mPa*sec), E10M (viscosity of 7500-14000 mPa*sec), K15M (viscosity of 13500-25200 mPa*sec), K100M (viscosity of 75000-140000 mPa*sec) or K200M (viscosity of 150000-280000 mPa*sec)), hydroxyethyl cellulose (such as HEC G (viscosity of 250-400 mPa*sec), HEC M (viscosity of 4500-6500 mPa*sec), HEC HX (viscosity of 3000-5000 mPa*sec), or HEC HHX (viscosity of 7000-11000 mPa*sec)), carboxymethyl cellulose (such as 7 MF (viscosity of 400-600 mPa*sec), 7H3F (viscosity of 2000-5600) mPa*sec), 7 HF (viscosity of 3000-5000 mPa*sec) or 7H4 (viscosity of 5000-9000 mPa*sec)), methylcellulose (such as A15C (viscosity of 1125-2100 mPa*sec), or A4M (viscosity of 3000-5600 mPa*sec)), polyethylene oxide, gelatin, a gum and a protein, and any mixture of at least two thereof.

In all aspects and embodiments of the present disclosure, the release modifying agent is comprised in an amount of at least about 30 mg per said dosage unit form, such as, for example at least about 30 mg, about 35 mg, about 40 mg, about 45 mg, about 50 mg and up to about 150 mg, 200 mg, 250 mg or 300 mg, 350 mg, 400 mg, 450 mg or 500 mg, for example 50-150 mg per dosage unit form, specifically wherein the dosage unit form is of total weight of from about 250 mg to about 2000 mg, such as, for example, from about 250 mg, 300 mg, 400 mg or 500 mg to about 1500 mg, more specifically from about 300 mg to about 1200 mg, and any range therebetween. In all these specific aspects and embodiments, the release adjusting agent is a hydrophilic polymer which when dissolved in water at 2% w/w gives a solution with viscosity of at least 150 mPa*sec, and the drug emulsifying agent is a lipid excipient having HLB greater than 6, or is a mixture of lipid excipients of which at least 50% have HLB greater than 6.

In all aspects and embodiments of the oral drug delivery device of the present disclosure, lipophilic drug or poorly water-soluble drug, or water insoluble drug, is a drug that has log P>2. Examples of lipophilic drugs are cannabinoids and their therapeutically active derivatives, including but not limited to Δ-9-tetrahydro-cannabinol (Δ9-THC, THC), iso-tetrahydrocannabinol (iso-THC), cannabinol (CBN) and cannabidiol (CBD) and pharmaceutically acceptable derivatives thereof, and pharmaceutically active metabolites thereof, as well as cannabis extract. Other cannabinoids are also referred to herein. The lipophilic drug according to the present disclosure may also be any one of ritonavir, fenofibrate, diclofenac, flurbiprofen diazepam, vitamin E, piroxicam, lopinavir, clofazimine, but not limited thereto.

Additional examples of cannabinoid therapeutically active derivatives are cannabigerol (CBG), cannabichromene (CBC), cannabielsoin (CBE), cannabicyclol (CBL), cannabicitran (CBT), cannabivarin (CBV), tetrahydro-cannabivarin (THCV), cannabidivarin (CBDV), and many others such as tetrahydrocannabidiol (THCBD), tetrahydrocannabigerol (THCBG), tetrahydrocannabichromene (THCBC), tetrahydro-cannabidivarol (THCBDV), cannabichromevarin (CBCV), cannabigerovarin (CBGV) and cannabigerol conomethyl ether (CBGM) and pharmaceutically acceptable derivatives thereof, and pharmaceutically active metabolites thereof.

In addition, a lipophilic or water-insoluble drug according to the present disclosure may be a non-lipophilic or water-soluble pharmaceutically active agent, which agent is rendered lipophilic by modification, specifically by chemical modification, for example hydrophobic ion pairing, wherein the modified active agent has log P>2. Such modified pharmaceutically active agents may be referred to herein as “modified drug” and the like. Non-limiting examples of pharmaceutically water soluble active agents that have been modified to become lipophilic or water-insoluble, having log P>2, are peptide drugs modified by pairing with a hydrophobic ion. Exemplary peptide drugs are Leuprorelin (LHRH agonist), insulin, desmopressin and others, such as for example therapeutic antibodies. Exemplary hydrophobic ions are anionic surfactants such as sodium docusate or sodium dodecyl sulfate, or fatty or sodium oleate. The hydrophobic ions pairing of the water soluble peptide drug with a hydrophobic ions, leads to increase of lipophilicity of the ion-paired peptides, the ion pair peptide product exhibiting increased Log P, thus capable of being emulsified in an emulsifying agent as disclosed herein.

In all aspects and embodiments of the present disclosure, the drug delivery device or dosage form thereof, may comprise from about 0.5 to about 500 mg, for example, from about 1 mg to about 300 mg, from about 5 to about 300 mg, from about 5 mg to about 300 or 400 mg, and any range therebetween of said lipophilic drug per device or unit dosage form.

In all aspects and embodiments of the present disclosure, the oral drug delivery device may comprise said at least one emulsified lipophilic drug at from about 1 to about 65% w/w, such as from about 10, 15, 20, 25, 30, 35 or 40% to about 50%, 55%, 60% or 65% w/w, for example, but not limited to from about 10 to about 65% w/w. The weight ratio lipophilic drug:emulsifying agent may be from about 2:1 to about 1:100, such as, for example, from about 1:1 to about 1:20 or 1:30.

With respect to the emulsifying agent/s, lipid excipients are generally characterized by their degree of dispersibility or solubilization capacity in aqueous media. The hydrophilicity of the lipid molecule, for example fatty acid ester, is affected in part by the type of polar group (glycerol, propylene glycol, polyethylene glycol, etc.) to which the fatty acid is esterified. The larger the polar group, the more hydrophilic the molecule becomes. The degree of esterification also impacts hydrophilicity of the lipid. Monoesters, for example, are more hydrophilic than diesters. As mentioned, an empirical system known as HLB is commonly used to categorize lipid excipients by the degree of affinity for the oily phase compared to aqueous phase in a formulation. In more detail, an HLB value of 1 applies to triglycerides (oily solubilizers). HLB value between 6 and 10 indicates the ability of a lipid/oily excipient to disperse in water. When above 10, the HLB value suggests an increasing degree of solubility in water. Briefly, the HLB of each excipient varies by type and distribution of hydrophobic and hydrophilic groups, degree of esterification such as monoesters compared to diesters, or tri-esters, and their quantities in the final excipient.

Lipid excipients as used herein may be oils, fatty acids, glycerides, polyoxylglycerides, polyglyceryl and polyalcohol esters, water insoluble surfactants, water soluble surfactants or co-solvents, such as, for example, coconut oil, cottonseed oil, soybean oil, castor oil, corn oil, olive oil, palm oil, peanut oil, peppermint oil, poppy seed oil, canola oil, hydrogenated oils, glyceryl esters of fatty acids, glyceryl behenate, glyceryl distearate, glyceryl laurate, glyceryl monooleate, glyceryl palmitate, glyceryl palmito stearate, glyceryl ricinoleate, glyceryl stearate, polyglyceryl oleate, polyglyceryl 10-tetralinoleate, behenic acid, caprylyic/capric glycerides, lauric acid, linoleic acid, linolenic acid, myristic acid, palmitic acid, palmitoleic acid, ricinoleic acid, stearic acid, soy fatty acids, oleic acid, tocopherols, Vitamin E, vitamin A, glyceryl esters, short-chain triglycerides, medium-chain triglycerides, long-chain triglycerides, pegylated fatty acids, phospholipids, sorbitan derivatives, hydrogenated castor oil derivatives, glycerin, polyglycolide glycerides, polyoxyethylene glycerides, polyethylene glycol-fatty acid esters, polyethylene glycol glycerol fatty acid esters, transesterfication products of oils and alcohols, polyglycerized fatty acids, polyglycerol fatty acid esters, propylene glycol fatty acid esters, mono and diglycerides, polyoxyethylene-polyoxypropylene block copolymers, oil PEG esters, hydrogenated oil PEG-esters, glycerol esters of fatty acids, saturated polyglycolized glycerides, polyoxylcastor oil, caprylate/caprate diglycerides medium chain (C8/C10) mono- and diglycerides, propylene glycol caprylate/caprate, propylene glycol dicaprylate/dicaprate, propylene glycol monolaurate, propylene glycol ricinoleate, propylene glycol monooleate, poloxamers (108, 124, 182, 183, 188,212, 217, 238, 288, 331, 338, 335, 407), sorbitan monoleate, d-C-tocopheryl polyethylene glycol 1000 succinate, polysorbates (20,60,80), and any mixture of at least two thereof.

In all aspects and embodiments of the presently disclosed oral drug delivery device, the emulsifying agent can be a lipid excipient having HLB (Hydrophilic Lipophilic Balance) greater than 6. lipid excipient with HLB value above 6 are, but not limited to, Gelucire 50/13 (stearoyl polyoxyl-32 glycerides), Gelucire 44/14 (lauroyl polyoxyl-32 glycerides), Gelucire 48/16 (polyoxyl-32 stearate (type I) NF), Labrafil 2125 (linoleoyl polyoxyl-6 glycerides), Labrafil 1944 (oleoyl polyoxyl-6 glycerides), Labrasol (caprylocaproyl polyoxyl-8 glycerides), Kolliphor RH40 (polyoxyl 40 hydrogenated castor oil), Kolliphor EL (polyoxyl-35 castor oil), Kolliphor HS 15 (macrogol-15-hydroxystearate), Kolliphor P 407/124/188 (poloxamer 407/124/188) and Kolliphor PS 80/60/20 (polysorbate 80/60/20), and any mixture of at least two thereof.

In some embodiments, the emulsifying agent is a mixture of lipid excipients of which at least 50% have HLB greater than 6, such as those listed above, and remaining lipid excipients are any one of oils, fatty acids, glycerides, polyoxyglycerides, polyglyceryl and polyalcohol esters or water insoluble surfactants, or any mixture of at least two thereof, wherein each said remaining lipid excipients has HLB lower than, equal to or higher than 6.

In all aspects and embodiments of the presently disclosed oral drug delivery device and dosage forms thereof, the hydrophilic film-forming polymer can be any one of povidone, copovidone, polyvinyl alcohol, hydrophilic polyacrylamide derivatives, proteins, gelatin, hydroxypropyl cellulose, polyethylene oxide, amino-methacrylate copolymer NF, hydroxypropyl methylcellulose, hydroxyethyl cellulose, carboxymethyl cellulose, sodium carboxymethylcellulose, methylcellulose, or polyvinyl alcohol-polyethylene glycol graft copolymer and any combination of at least two thereof.

In all aspects and embodiments of the presently disclosed oral drug delivery device and dosage forms thereof, the film-forming polymer and the release-modifying agent together constitute from about 25 to about 85% w/w, such as from about 25 to 70% w/w, more specifically from about 30 to about 70% w/w of said device. In all aspects and embodiments of the present disclosure, the drug delivery device optionally comprises a plasticizer at up to 20% w/w, preferably at up to 15% w/w, more preferably at from about 5% to about 10% w/w. Exemplary plasticizing agents (plasticizers) optionally used in the oral drug delivery device of the present disclosure and dosage unit forms thereof are, but not limited to, polyethylene glycols, citrate esters, phthalate esters, glyceryl esters, short-chain triglycerides, medium-chain triglycerides, long-chain triglycerides, olive oil, hydrogenated castor oil, triacetin, glyceryl stearate, glyceryl behenate, dibutyl sebacate, aliphatic alcohols, fatty acids, pegylated aliphatic alcohols and pegylated fatty acids, phospholipids, sorbitan derivatives, polysorbates, poloxamers, hydrogenated castor oil derivatives, glycerin, propylene glycol, and any combination of at least two thereof.

In some aspects and embodiments of the oral drug delivery device of the present disclosure and dosage unit forms thereof the device/dosage unit form are in the form of an oral pharmaceutical tablet. Such tablets can generally comprise tableting excipients or additives, which can be, but are not limited to, binders, diluents/fillers, lubricants and glidants.

Binders are materials that bind the tablet ingredients together, giving shape and mechanical strength to powdered materials used in tablet manufacture. Binders ensure that the tablets remain intact after compression, as well as improve the free-flowing qualities of powdered materials without retarding disintegration or dissolution. Binders are used alone or in combination with one another. They are added as dry powders or in the form of their solution in a suitable and appropriate solvent. Materials commonly used as binders include acacia gum, hydroxypropyl methylcellulose, polyvinyl pyrrolidone (PVP), corn starch, microcrystalline cellulose, etc.

Diluents/Fillers are added to increase the size of the tablet, so as to obtain a significant tablet weight that can be handled or compressed. Fillers should be chemically inert, non-hygroscopic, hydrophilic and must exhibit good compression properties. Lactose is a common filler used in formulations. Other examples of bulking agents used in tablet manufacture include mannitol, dicalcium phosphate, calcium sulfate, dry starch, cellulose, kaolin, sodium chloride, anhydrous lactose, sorbitol, sucrose, etc.

Lubricants reduce friction between the powder mix and the die walls during compression and ejection. They also prevent the mixed powders/granules from sticking to the processing zone of the tablet press especially the punches and die. Materials commonly used as lubricants are polyoxyethylene stearates, and lauryl sulphate salts. Magnesium stearate, glyceryl behenate, stearic acid, glyceryl palmitostearate, etc.

Glidants are fine powders that improve the flow of the blend during tablet manufacturing by reducing friction and adhesion between particles. They enhance the movement of powders or granules within the hopper and into the die cavity prior to compression. By enhancing flow rates there is less weight variability of the tablets manufactured, resulting in more consistent dosing of the drug substance. Examples of glidants used in tablet manufacture include colloidal silicon dioxide, talc, corn starch, etc.

In all aspects and embodiments of the oral drug delivery device of the present disclosure and dosage unit forms thereof, all constituents, namely hydrophilic film-forming polymers, CR and IR release modifying/adjusting agents, lipid excipients (emulsifying agents) and tableting excipients are pharmaceutically and physiologically acceptable.

Representative methods for producing oral drug CR delivery devices and dosage units thereof are presented in the following examples.

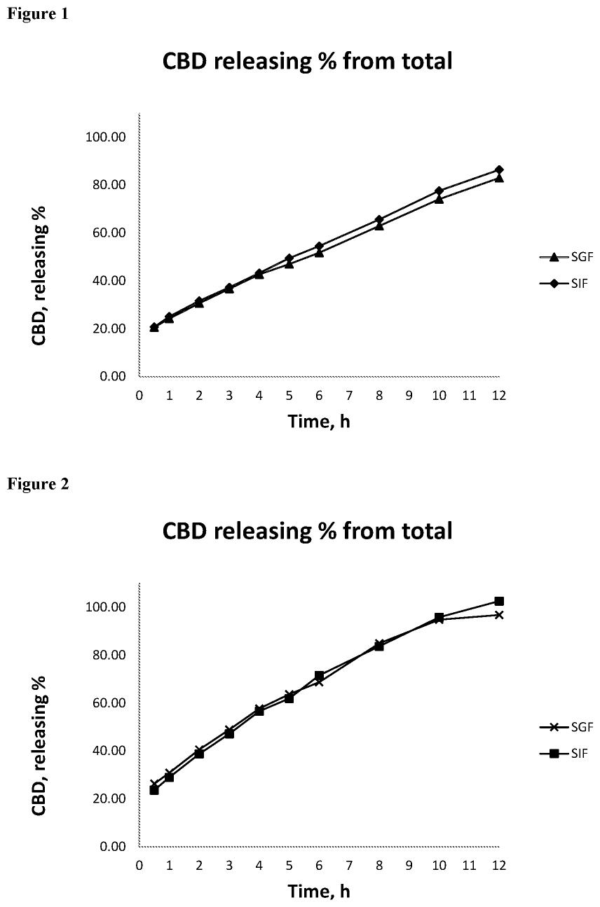

In some specific embodiments, the emulsified drug, the film forming-polymer, the release modifying polymer and the optional plasticizer are all comprised in a drug-containing laminated member of the drug delivery device, comprising identical layers in which the drug is embedded. The preparation of exemplary such drug delivery devices and dosage unit forms thereof, comprising CBD as an exemplary lipophilic drug as defined herein, is presented in Examples 1-3. Generally, the drug is emulsified by dissolving in (mixing with) a suitable emulsifying agent. The solution is added to water or optionally an aqueous solution of a plasticizer, thereby forming a self-emulsion (micelles) of the drug. Suitable film-forming polymer/s and release modifying agent/s are added to the drug emulsion and mixed until dissolved. The final emulsion is cast on suitable support, and dried. The product is a film, which can be cut into units; units are laminated, processed into a suitable configuration, and fitted into an oral capsule. An immediate release fraction is prepared separately (in Examples 1-3 in powder form), and introduced into the capsule. The tested dissolution profile was similar in SGF and SIF. Following immediate release of the IR position of the drug, dissolution continued for at least 12 hours, as shown in FIGS. 1-3.

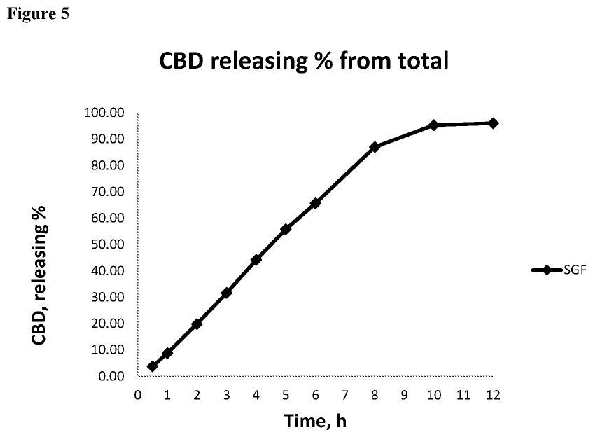

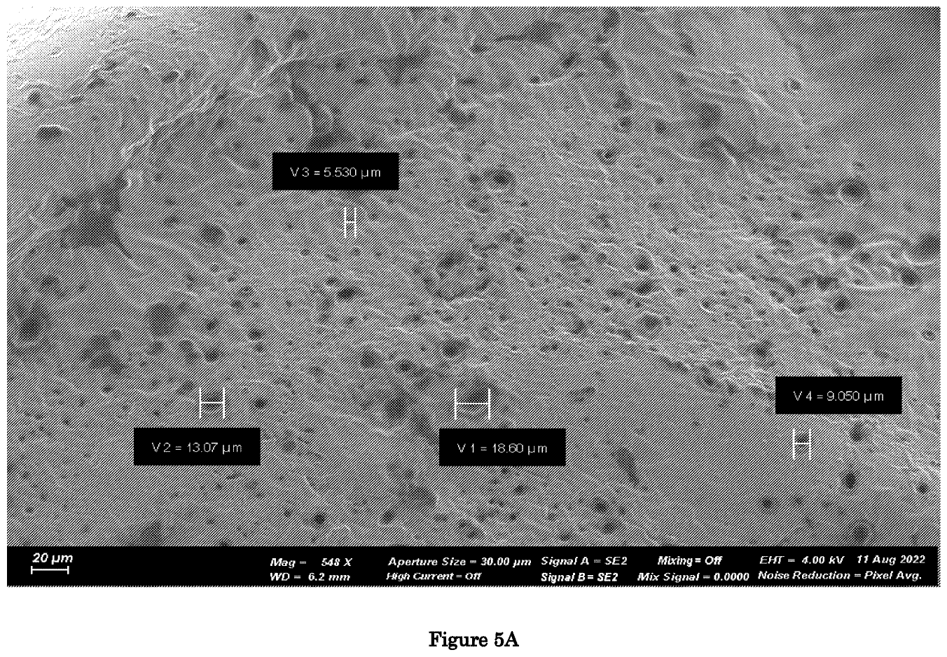

In other embodiments, the drug containing member of a drug delivery device according to the present disclosure is prepared by hot melt extrusion, as shown in Example 5. Generally, the drug (e.g. CBD) is emulsified by dissolving in (mixing with) a suitable emulsifying agent. Suitable film-forming polymer/s, release modifying agent/s and optional plasticizer are all added to the drug emulsion, and all ingredients are blended. The blend is introduced to a hot melt extrusion machine and processed. The final product is a uniform extrudate comprising the emulsified drug. Extrudates can be produced with suitable diameters, for example 7-8 mm diameter, cut into a suitable length and inserted into an oral capsule. As shown in FIG. 5, controlled release of the active drug (CBD) lasted about 10 hours. An IR formulation, in liquid or powder form can be introduced into the capsules containing the delivery device produced by the exemplified hot melt technique, same as in Examples 1-3.

In other embodiments, the drug delivery device according to the present disclosure comprises a drug-containing member, and a controlled release (CR) member. The preparation of this type of delivery device is presented in Example 4. A drug-containing member (layer) is generally prepared by dissolving the drug in (mixing the drug with) a suitable emulsifying agent. The solution is added to water or optionally an aqueous solution of a plasticizer, thereby forming a self-emulsion (micelles) of the drug. Suitable film-forming polymer/s is/are to the drug emulsion and mixed until dissolved. The final emulsion is cast on suitable support, and dried. The product is a film. A drug-free CR layer is prepared separately, by dissolving or dispersing a release modifying agent, optionally together with suitable amounts of a film-forming polymer and/or a plasticizer, in a suitable solvent. The mixture is cast on suitable support, and dried. The dried product, which is a film, is laminated on the drug-containing film, and the laminated films are rolled together to give an essentially cylindrical laminated body comprising two distinct layers, drug-containing layer and CR layer. The cylindrical body can then be cut into cylindrical segments of suitable length. Optionally, the drug-containing film and the CR film may be cut into similar or different units before lamination, and a unit of drug-containing film and a unit of the CR film are laminated and rolled together into an essentially cylindrical laminated body comprising two distinct layers, drug-containing layer and CR layer. In the specific embodiment presented in Example 5, the CR layer is innermost. If desired, the CR layer can be outermost. The direction of rolling the laminated layers can be used for specific drug release profiles. The resulting cylindrical segment or cylindrical body is introduced into an oral capsule. If desired, an IR formulation, in powder or liquid form may be added to and/or coated onto the capsule. As shown in Example 4 and FIG. 4, the release profile in GSF demonstrated prolonged and controlled release profile for up to 10 hours.

In other embodiments, the drug delivery device according to the present disclosure may be formed as an orally administrable pharmaceutical controlled release tablet. An exemplary preparation of a tablet in accordance with the present disclosure, in which the active pharmaceutical ingredient is CBD and the release modifying agent is added to the drug emulsion, is presented in Example 12. Thus, for example, a tablet formulation is prepared by dissolving the drug in emulsifying agent/s. The mixture is added to an aqueous medium optionally comprising a plasticizer, to form a self-emulsion. Release modifying agent and a hydrophilic film forming polymer are added, until all are dissolved. The final emulsion is cast on a suitable support and dried. The dried product, for example a film, is milled and sieved using a suitable sieve (e.g. 500-micron sieve). Tableting additive/s are added and blended, and the blend is pressed into tablets using suitable press machine.

An alternative method for preparing tablets is presented in Example 13, in which the release modifying agent is added as a powder to the tablet mixture. In this example, THC was used as the active pharmaceutical agent. THC is dissolved in in emulsifying agent/s. The mixture is added to an aqueous medium optionally comprising a plasticizer, to form a self-emulsion. A hydrophilic film forming polymer is added ( ) and ingredients are mixed until all are dissolved. The final emulsion is cast on a suitable support and dried. The dried product, for example a film, is milled and sieved using a suitable sieve (e.g. 500-micron sieve). Tableting additive/s are added together with a release modifying agent, and all are blended. The blend is pressed into tablets using suitable press machine.

Still alternatively, a tablet may be prepared by hot melt extrusion, as presented in Example 14. CBD is used as the active agent. A hot melt extrudate is prepared as described above and in Example 5. The extrudate is milled and sieved using a suitable sieve (e.g. 500-micron sieve). Tableting excipients and a release modifying agent are added. All ingredients are blended. The blend is pressed into tablets using suitable press machine.

In all aspects and embodiments of the present disclosure, the disclosed oral drug delivery device or pharmaceutical dosage unit forms thereof in which the drug is at least one cannabinoid or cannabis extract, can be used in methods for any one of treating, alleviating and preventing worsening of a disease, disorder or condition responsive to cannabinoid therapy in a subject in need, said method comprising orally administering to said subject said drug delivery device or pharmaceutical dosage unit form thereof. The disease, disorder or condition responsive to cannabinoid therapy can be any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases such as Crohn's disease and colitis, post-traumatic stress disorder (PTSD) or epileptic seizures, Parkinson's disease, spinal cord injury, fibromyalgia, Alzheimer's disease and dementia or any other condition responsive to cannabinoid therapy.

The present disclosure also relates to a method of increasing the absorption time of an active pharmaceutical ingredient (API) having log P>2 in a subject in need thereof, by administering to said subject a device or a pharmaceutical dosage unit form according to all described aspects and embodiments thereof.

In all aspects and specific embodiments of the presently disclosed subject matter, methods of treatment and therapeutic uses of the presently disclosed oral drug delivery devices and pharmaceutical dosage unit forms thereof can comprise administration of the dosage form once, twice or three times a day, or at lower frequencies such as, for example, once or twice weekly. Further, administration can be under fed as well as fasted conditions.

In all aspects and embodiments, the present disclosure further provides for specific oral delivery devices for lipophilic or poorly water-soluble drugs, including but not limited to cannabinoids as herein defined, and dosage forms thereof, in which the drug is in emulsified form or is mixed with at least one emulsifying agent. The disclosed lipophilic drugs formulations can provide stable plasma levels of the drug for longer duration, prolonging drug availability, thereby leading to potential improvements in efficacy of the active principal. The proposed drug delivery devices and dosage unit forms thereof can generate a continuous and effective exposure of target organs and tissues to the drug, the formulation releasing the drug in the stomach and thereafter in the duodenum and intestines for a prolonged time, such as, for example, from several hours following administration to about 12, and up to 24 hours following administration, usually from about 3 hours, and up to any one of 4, 5, 6, 7, 8, 9, 10, 11, 12 and up to 15 hours. The disclosed drug delivery devices and dosage unit forms thereof can improve the efficacy of treatment, while reducing the number of daily doses. The proposed drug delivery devices and dosage unit forms thereof provide for use of a predetermined dose of the active drug. Importantly, the drug delivery device in accordance with all aspects and embodiments of the present disclosure can be versatile in term of the rate of release of the active ingredient. Mainly, while the release is controlled release, its rate can be controlled by specific features of the delivery device disclosed herein. Such features can be the load of the drug, the amount of the modifying release agent, the features of the drug emulsion or mixture with the emulsifying agent, such as the specific emulsion formulation, the API to emulsifying agents ratio, micelles size, the geometry and structure of the device, or layers thereof, where present, as detailed below and exemplified in the following Examples, Controlled release can be preceded or accompanied by immediate release of components contained in the disclosed dosage unit forms of the disclosed drug delivery device.

Also disclosed herein are pharmaceutical dosage forms, for example dosage unit forms of cannabinoids. Essentially, a pharmaceutical dosage form as disclosed herein comprises a controlled release cannabinoid/s delivery device, integrated into an oral pharmaceutical capsule, which may be a hard or soft gel capsule.

The present disclosure further provides for specific drug delivery devices and dosage unit forms thereof for cannabinoids, namely orally administered CR and/or combined CR and IR delivery devices, or dosage unit forms thereof, of cannabinoids as disclosed herein. The disclosed cannabinoid formulations can provide stable plasma levels of the cannabinoid drug for longer duration, prolonging drug availability, thereby leading to potential improvements in efficacy of the active principal. As mentioned, the proposed cannabinoid delivery devices and dosage unit forms thereof can generate a continuous and effective exposure of target organs and tissues to the drug, can improve the efficacy of treatment, while reducing the number of daily doses, can provide for use of a predetermined dose of the active cannabinoid ingredient/s, and can be versatile in term of the rate of release of the active ingredient.

### Definitions

The terms “drug”, “active substance”, “API” (Active Pharmaceutical Ingredient) or “active principal” or “active ingredient” or “active agent”, used herein interchangeably, refer to a pharmaceutically active substance that provides a therapeutic/physiological effect to a patient, and can also refer to a mixture of at least two thereof.

The terms “lipophilic drug” or “poorly water-soluble drug” or “lipophilic modified drug” or “poorly water-soluble modified drug” as used herein interchangeably refer to a drug which is insoluble or only slightly soluble in water or a drug that is chemically modified to become insoluble or only slightly soluble in water, and specifically refer to “drug having a Log P>2” or “a modified drug having a Log P>2” or “drug modified, specifically chemically to have a Log P>2”, which as used herein interchangingly refer to drugs or modified drugs of which the logarithm of the partition coefficient, defined as a particular ratio of the concentrations of a solute between water and octanol, is greater than 2.

The terms “cannabinoid”, “cannabinoid derivative” or “cannabinoid metabolite”, as used interchangeably herein, refer to any active principal of cannabis. The “cannabinoid” as defined herein can also be a cannabimimetic, not derived from cannabis. The terms “cannabinoid”, “cannabinoid derivative” or “cannabinoid metabolite”, as used interchangeably herein, refer also to any mixture of at least two cannabinoids, as defined herein.

The terms “cannabis extract” or “cannabis concentrate” as used interchangeably herein, refer to an extract of the cannabis plant which contains cannabinoids and optionality terpene/s and/or other compounds.

The terms “drug-releasing formulation” as used herein refer to any composition of matter which comprises a drug and releases the drug following administration to a subject.

The terms “emulsified drug” or “a drug emulsified in an emulsifying agent” or “a drug emulsion” or “an emulsion of a drug in an emulsifying agent” or “a drug in emulsified form” or the like, used herein interchangeably, refer to poorly water-soluble drug/s which is/are dissolved in or mixed with emulsifying agent/s, and upon dilution in aqueous medium such as gastric fluid (GF), intestinal fluid (IF) or gastrointestinal fluid (GIF), can form fine oil in water (o/w) emulsions or microemulsions. When referring to defined amounts/quantities of “emulsified drug” or “a drug emulsified in an emulsifying agent” or “a drug emulsion” or “an emulsion of a drug in an emulsifying agent” or “a drug in emulsified form” or the like, used herein interchangeably, the amount indicates the combined amounts of both the drug and the emulsifying agent/s. Thus, the terms “drug emulsion” and “mixture of drug with emulsifying agent/s” are both used herein.

The terms “an emulsifying agent” or “emulsifier” as used interchangeably herein refer to a compound or substance that concentrates at the interface of two immiscible phases, usually an oil, and water. It lowers the interfacial free energy, reduces the interfacial tension between the phases, and forms a film or barrier around the droplets of the immiscible, discontinuous phase as they are formed, preventing the coalescence of the droplets. The term “emulsifying agent” includes, but is not limited to oils, glycerides, water insoluble surfactants, water soluble surfactants or co-solvents or any mixture of at least two thereof, as described in detail herein.

As used interchangeably herein, “dosage units”, “dosage forms”, “oral dosage units”, “dosage unit forms”, “oral dosage unit forms” and the like refer to solid dosage forms as known in the art and may be used herein interchangeably. The dosage forms are intended for peroral use, i.e., to be swallowed (ingested) by a patient/subject in need thereof. The terms “oral capsule” and “peroral capsule” are used herein interchangeably.

The term “solid” as used herein with reference to all aspects and embodiments of disclosed drug delivery device and dosage form thereof, and with reference to their various components, such as drug-containing component (or layer or member) controlled release (CR) component (or layer or member), immediate release (IR) component (or layer or member or powder), or various mixtures of components (such as but not limited to a mixture of the active drug and emulsifying agent), is to be taken to mean a composition or article of manufacture which is solid at room temperature, which is malleable or pliable, into a desired form. The terms “solid”, “malleable solid” and “pliable solid” are used herein interchangingly.

The term “drug delivery device” or similar terms, as may be used interchangeably herein, refer to a component of the disclosed dosage units, comprising at least one emulsified drug. The “drug delivery device” can be formed as a dosage unit form, as described herein.

“Controlled-release” (CR) or “extended release” as used herein denotes a manner IN which a dosage form, dosage unit form or drug delivery device and the like, releases a drug, for example but not limited to a cannabinoid, at a controlled rate over extended designable time intervals, specifically predetermined extended time intervals, at needed quantities to achieve a desired drug, for example but not limited to cannabinoid, serum level, and produce a prolonged or sustained pharmacological effect.

The terms “release modifying agent” and “release adjusting agent” as used herein interchangeably, are generally polymeric agent, which lead to release of a drug from a drug-delivery device or component thereof or dosage form thereof in a manner that is different from release in their absence.

The phrases “prolonged period/s”, “extended time interval/s” and the like, as used interchangeably herein in connection with controlled release, refer to a period of delivery of at least 80% of the drug dose contained in the dosage unit or drug delivery device that lasts for from several hours following administration to about 12, and up to 24 hours following administration, usually from about 3 hours, and up to any one of 4, 5, 6, 7, 8, 9, 10, 11, 12 and up to 15 hours.

The phrase “immediate release” (IR) as used herein denotes a manner that a dosage form or drug delivery device releases the drug immediately upon exposure to or contact with gastric medium. By “immediately upon exposure to gastric fluid” is to be understood release within up to 2 hours from said exposure or contact. Specifically, immediate release of a drug is within about 30 to about 60 minutes from exposure to or contact with gastric fluid. The term “immediate release (IR) component” as used herein is to be taken to mean a component that releases the drug therefrom in this described manner. The IR component used herein can be in the form of a coating (e.g. on a capsule as described herein) or a powder (e.g. within the capsule as described herein) or in any other suitable form.

The term “stable” plasma or serum levels of a drug, for example but not limited to, a cannabinoid as herein defined or active derivative or metabolite thereof is to be taken to mean a therapeutically effective plasma level of the active cannabinoids and/or active metabolites thereof over suitable period of time. Specifically, stable plasma levels can, for example, refer to continuous therapeutically effective levels, steady state levels, and the like.

The term “AUC” as known in the art and used herein refers to the Area Under the Curve (mathematically known as the definite integral) in a plot of drug concentration in blood plasma vs. time. In practice, the drug concentration is measured at certain discrete points in time and the trapezoidal rule is used to estimate AUC. The AUC (from zero to infinity) represents the total drug exposure over time. The term Cₘₐₓ is the maximum (or peak) serum concentration that a drug achieves in a specified compartment or test area of the body after the drug has been administrated. The related pharmacokinetic parameter tₘₐₓ is the time at which the Cₘₐₓ is observed.

“Simulated gastric fluid” (“SGF”) and “Simulated intestinal fluid” (“SIF”) as used herein, refer to “Gastric fluid, Simulated, TS” and “Intestinal fluid, Simulated, TS” solutions as defined by the United States Pharmacopeia 30 National Formulary, without the corresponding enzymes.

“Gastric medium”, “gastric fluid” and “intestinal medium” as used interchangeably herein denote a biological medium of the stomach and intestines, respectively, or an artificial medium, used to mimic the environment of the stomach or intestines, exemplified but not limited to, “Simulated gastric fluid” (“SGF”) and “Simulated intestinal fluid” (“SIF”).

The term “aqueous medium” as used herein denotes liquid media, based on water, specifically “gastric medium”, “gastric fluid”, “intestinal medium”, as defined above, and distilled water.

The term “biodegradable” or “degradable”, and “bio-erodible” or “erodible” as used herein is intended as capable of being biochemically, chemically and/or physically processed, reduced or broken down in the body of a patient, within a time period between several seconds to several days from ingestion of the dosage unit form or delivery device. The terms “completely degraded/biodegraded”, “essentially completely degraded/biodegraded”, or “completely eroded/bio-eroded” or used interchangingly herein mean at least 70%, 80%, 90% and up to 100% degradation/erosion and disappearance of the delivery device or dosage form.

The terms “inert” or “inactive” or “inactive ingredient” or “inert ingredient” or “inactive excipient” or “inert excipient”, as used interchangeably herein refer to components of the delivery device or dosage unit form that do not instantly react with the drug or active ingredient or adversely affect its properties, or cause any biological effect upon administration to a subject when administered in reasonable amounts to said subject. General examples of these components are described in “The Handbook of Pharmaceutical Excipients”, 4^{th} Edition, by Rowe, Sheskey and Weller, Pharmaceutical press, 2003. Additional exemplary list is Inactive Ingredients Guide of the Food and Drug Administration, USA.

The terms “film” and “layer”, sometimes preceded by the term “polymeric”, are used interchangeably herein in connection with some or all components of the drug delivery devices, laminated drug delivery device and their dosage unit forms, their preparation and use as described herein, as context dictates.

The terms “drug-containing layer” “drug-containing component” or “drug-containing polymeric component” as used herein are to be taken to mean a polymeric film or polymeric layer comprising at least one polymer and at least one drug, respectively a cannabinoid, as defined herein.

The terms “emulsified drug-containing layer” “emulsified drug-containing component” or “emulsified drug-containing polymeric component” as used herein, are to be taken to mean a polymeric film or polymeric layer or polymeric member or component comprising at least one polymer and at least one drug, respectively a cannabinoid, in emulsified form. These terms can relate component/s or layer/s of the delivery device and also to IR layers of the delivery device or the dosage unit form.

The term “polymer” as used herein denotes a polymeric material as known in the art and described in greater detail below, but is also sometimes used, in context of more general aspects, to encompass the polymeric composition of one or more polymers, and optionally at least one plasticizer; i.e., the inert materials of polymeric layer, members or components comprised in the disclosed drug delivery device and/or the dosage unit form/s thereof.

The term “polymeric component” or “drug-containing polymeric component/member/film/layer” as used herein is to be taken to mean that part of the drug delivery device in which the drug, and optionally other pharmaceutically active agent/s, are comprised.

The term “lamination” and the like as used herein, unless the context clearly indicates otherwise, means in reference to two and more polymeric components, members, films or layers, the action of layering, stacking, filing, piling, superimposing at least one polymeric component/member/film/layer over at least one additional polymeric component/member/film/layer, bringing them into a tight contact, and under suitable conditions of temperature and ambience, applying pressure sufficient to produce a uniform structure wherein boundaries the individual layers are no longer readily distinguishable, or the individual layers are irreversibly bound to the contiguous layers. The lamination is usually between polymeric sheets of similar to identical composition, and similar thickness, although films of significantly different thickness but similar or identical composition may also be laminated. In addition, the lamination may be also performed on a plurality of films of different composition, if at least two of such films are similar or identical in composition and/or thickness, and if in the obtained structure the boundaries of the individual layers are not readily distinguishable or the individual layers are irreversibly bound to contiguous layers. The term “readily distinguishable” should be understood as being within the aptitude of a person skilled in the art to distinguish the individual layers without application of microscopy or spectrometry.

A “patient” or “subject” as referred to herein is an animal, specifically a human, who may be administered with the drug delivery device disclosed herein and dosage units thereof.

The term “treat” and forms thereof such as “treatment” or “treated” as used herein is to be taken to mean, to prevent a symptom (e.g., pain or nausea), or prevent worsening, or arrest, or alleviate, or improve or cure the patient's disease or condition or symptom associated therewith.

The term “ratio” as used herein, unless otherwise indicated, refers to a weight ratio (also designated w/w)

The term “suitable” as used herein is to be taken to mean having the properties that enable providing the defined result.

“About” as used herein generally refers to approximate values. When referred to a dose of a drug in milligrams, “about” should be understood as including the range of a value±15%. When referred to other values, the term should be understood as including the range of a value±15%, for example ±15%, ±12%, ±10%, ±8%, ±5%, ±2% or ±1%.

As used in the description and claims, the forms “a”, “an” and “the” include singular as well as plural references unless the context clearly dictates.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### DESCRIPTION OF NON-LIMITING EXAMPLES

### Example 1: CBD 5 mg IR and 20 mg CR Formulation 1

### CR Fraction:

An exemplary preparation of an emulsion containing CBD and the preparation of an CR (controlled release) unit dosage form containing CBD micelles, is presented below. The composition of a single unit is summarized in Table 1.

TABLE 1

| CR formulation 1 | mg per unit | % In formulation |
|---|---|---|
| CBD | 20 | 3.2 |
| Peceol | 42 | 6.8 |
| Kolliphor EL | 98 | 15.8 |
| Klucel EF | 275 | 44.4 |
| Klucel GF | 135 | 21.8 |
| PEG 400 | 50 | 8.1 |
| Total | 620 | 100 |

CBD was dissolved in a Peceol and Kolliphor mixture heated to 70° C. using magnetic stirrer to obtain a clear solution.

In a 0.2-Liter planetary mixer, PEG 400 was dissolved in water. The CBD clear solution was added to form a self-emulsion. Klucel EF and Klucel GF were added, and dissolved for about 90 minutes in the aqueous mixture.

The final emulsion was cast on a PET (Mylar™) web, using a tabletop casting machine with a knife space of 950 μm. The cast emulsion was dried in an oven at 60° C. for about 60 minutes. The dried product was a film with a thickness of about 120 μm.

6 sheets of the dry film were stacked one on top of the other and laminated manually to give a laminated film, having a thickness of about 750 μm.

The laminated film was punched into 21×45 mm rectangular units, each with weight of about 600-650 mg, each comprising 20 mg CBD.

Each unit was rolled into cylindrical shape, and each cylinder was inserted into a 00 hard gelatin capsule.

### IR Fraction

An exemplary preparation of a lipid-based powder, containing CBD for IR (immediate release), is presented below. The materials used for preparing the powder formulation and the quantities present in a single capsule containing the powder unit are summarized in Table 2.

TABLE 2

| Powder Formulation 1 | mg per unit |
|---|---|
| CBD | 5 |
| Peceol | 6 |
| Kolliphor EL | 14 |
| AVICEL 102 | 75 |

CBD was dissolved in Peceol and Kolliphor mixture heated to 70° C. using a magnetic stirrer to obtain a clear solution.

Avicel 102 was added to the solution and mixed for about 30 minutes to obtain a uniform powder.

100 mg powder containing 5 mg CBD were inserted into the capsule containing the CR fraction to give a final unit dosage form.

Each final unit contains 5 mg IR CBD and 20 mg CR CBD

The dissolution profiles of the final units were tested.

### Dissolution Profile:

The dissolution of the capsules containing the IR and the CR fractions was tested using the following method:

### Dissolution Parameters:

Medium:
Simulated Gastric Fluid (SGF), pH 1.2 or
Simulated Intestinal Fluid (SIF), pH 6.8
Temperature: 37±0.5° C.
Apparatus: 20 Mesh baskets at 100 RPM
Volume: 900 ml
Sampling time points: 0.5; 1; 2; 3; 4; 5; 6; 8; 10; 12 hours

At each sampling point, 5 ml aliquot of the solution was sampled from the vessel. The samples were tested using HPLC method to analyze CBD content.

### HPLC Parameters:

| Instrument | HPLC with an auto-injector | |
|---|---|---|
| Column | Gemini NX, C18, 5μ, (50 × 4.6)mm | |
| | (or equivalent) | |
| Column temperature | 30° | C. |
| Sample temperature | 5° | C. |
| Mobile Phase | Purified water: Acetonitrile (35:65) | |
| Flow Rate | 2.0 | mL/min |
| Run Time | Not less than 6.5 minutes | |
| Detection | UV at 210 nm | |
| Injection volume | 40 | μL |

| | |
|---|---|
| Diluent | SGF/SIF:Ethanol (1:1) |
| Needle wash | Methanol |

The results of the dissolution test of Example 1 are presented in FIG. 1. As can be seen, the CBD release profile is similar in both SGF and SIF. The release profile is linear profile demonstrating prolonged and controlled release profile up to 14 hours.

### Example 2: CBD 5 mg IR and 20 mg CR Formulation 2

### CR Fraction:

An exemplary preparation of an emulsion containing CBD and the preparation of an oral CR dosage form containing CBD micelles, is presented below. The composition of a single unit is summarized in Table 3.

TABLE 3

| CR formulation 2 | mg per unit | % In formulation |
|---|---|---|
| CBD | 20 | 3.2 |
| Peceol | 42 | 6.8 |
| Kolliphor EL | 98 | 15.8 |
| Klucel EF | 345 | 55.7 |
| Klucel GF | 65 | 10.5 |
| PEG 400 | 50 | 8.1 |
| Total | 620 | 100 |

CBD was dissolved in Peceol and Kolliphor mixture heated to 70° C. using magnetic stirrer to obtain a clear solution.

In a 0.2-Liter planetary mixer, PEG 400 was dissolved in water.

The CBD clear solution was added to form a self-emulsion. Klucel EF and Klucel GF were added, and dissolved for about 60 minutes in the water.

The final emulsion was cast on a PET (Mylar™) web, using a tabletop casting machine with a knife space of 1000 μm. The cast emulsion was dried in an oven at 60° C. for about 60 minutes. The dried product is a film with a thickness of about 150 μm.

5 sheets of the dry film were stacked one on top of the other and laminated manually.

The laminated films had a thickness of about 750 μm.

The laminated film was punched into 21×45 mm rectangular units, each with weight of 600-650 mg, each unit comprising 20 mg CBD.

The units were rolled to cylinder shape, and each cylinder was inserted into a 00 hard gelatin capsule.

### IR Fraction

The IR fraction is the same as Example 1.

Each final unit contains 5 mg IR CBD and 20 mg CR CBD.

The final units were tested to define the dissolution profile using the same method and parameters as described in Example 1.

The results of the dissolution test of Example 2 are presented in FIG. 2. As can be seen in FIG. 2, the CBD release profile is similar in both SGF and SIF. The release profile is linear profile demonstrating prolonged and controlled release profile up to 12 hours.

### Example 3: CBD 25 mg IR and 225 mg CR Formulation 3

### CR Fraction:

An exemplary preparation of an emulsion containing CBD and the preparation of an oral CR dosage form containing CBD micelles, is presented below. The composition of a single unit is summarized in Table 4.

TABLE 4

| CR formulation 3 | mg per unit | % In formulation |
|---|---|---|
| CBD | 225 | 30.2 |
| Lauroyl Polyoxyl-32 | 225 | 30.2 |
| glycerides | | |
| CMC 7L2P | 135 | 13.4 |
| HPMC K1500 | 100 | 18.1 |
| POLOXAMER 407 | 60 | 8.1 |
| Total | 745 | 100 |

CBD was dissolved in Lauroyl Polyoxyl-32 glycerides heated to 70° C. using magnetic stirrer to obtain a clear solution.

In a 0.2-Liter planetary mixer, POLOXAMER 407 was dissolved in water.

The CBD clear solution was added to form a self-emulsion. CMC and HPMC were added, and dissolved for about 60 minutes in the water.

The final emulsion was cast on a PET (Mylar™) web, using a tabletop casting machine with a knife space of 1100 μm. The cast emulsion was dried in an oven at 60° C. for about 60 minutes. The dried product was a film with a thickness of about 150 μm.

5 sheets of the dry film were stacked one on top of the other and laminated manually.

The laminated films have a thickness of about 750 μm.

The laminated film was punched into 21×45 mm rectangular units, each with weight of 720-780 mg, each unit comprising 225 mg CBD.

Each unit was rolled into cylindrical shape, and each cylinder was inserted into a 00 hard gelatin capsule.

### IR Fraction

An exemplary preparation of a lipid-based powder, containing CBD for IR, is presented below. The materials used for preparing the powder and the quantities present in a single capsule containing the powder unit are summarized in Table 5.

TABLE 5

| Powder Formulation 2 | mg per units |
|---|---|
| CBD | 25 |
| Kolliphor EL | 25 |
| AVICEL 102 | 70 |

CBD was dissolved in Kolliphor EL heated to 70° C. using a magnetic stirrer to obtain a clear solution.

Avicel 102 was added to the solution and mixed for about 30 minutes to obtain a uniform powder.

120 mg powder containing 25 mg CBD was inserted into the capsule containing the CR fraction.

Each final unit contains 25 mg IR CBD and 225 mg CR CBD

The final units were tested to define the dissolution profile.

### Dissolution Profile:

The dissolution of the capsules containing the IR and the CR fractions was tested using the same method and parameters as described in Example 1.

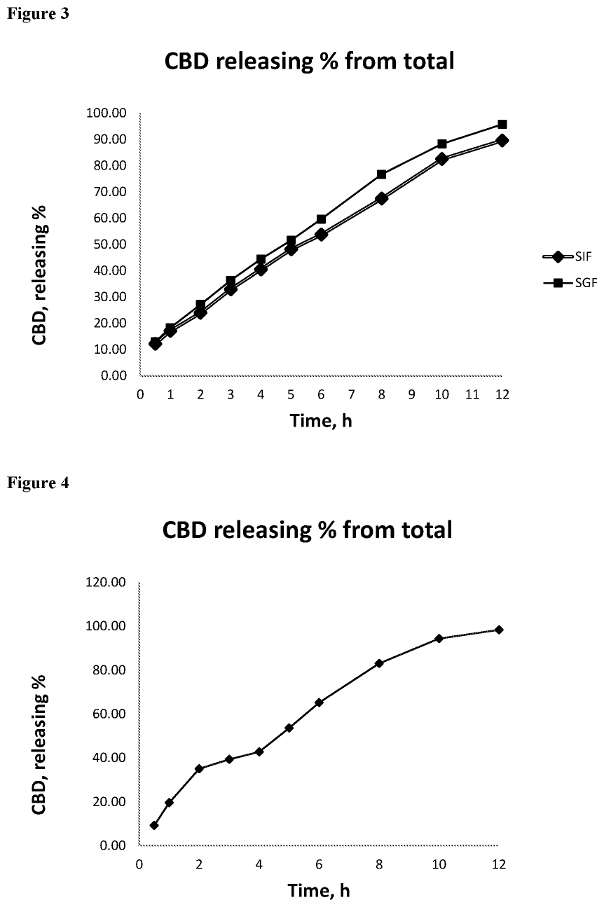

The results of the dissolution test of Example 3 are presented in FIG. 3. The CBD release profile is similar in both SGF and SIF. The release profile is linear profile demonstrating prolonged and controlled release profile up to 12 hours.

### Example 4: CBD 10 mg CR Formulation 4-Separate Layers

An exemplary preparation of an oral CR dosage form containing CBD micelles, is presented below. Each unit contains 2 different layers which laminated together to form the final unit: Drug layer and CR layer

### Drug Layer:

An exemplary preparation of an emulsion containing CBD and the preparation of a dry film containing CBD micelles, is presented below. The composition of a single unit is summarized in Table 6.

TABLE 6

| Drug layer (without | | |
|---|---|---|
| CR) formulation 1 | mg per unit | % In formulation |
| CBD | 10 | 1.9 |
| Peceol | 42 | 7.9 |
| Kolliphor EL | 98 | 18.5 |
| Klucel EF | 350 | 66.0 |
| PEG 400 | 30 | 5.7 |
| Total | 530 | 100 |

CBD was dissolved in Peceol and Kolliphor mixture heated to 70° C. using magnetic stirrer to obtain a clear solution.

In a 0.2-Liter planetary mixer, PEG 400 was dissolved in water. The CBD clear solution was added to form a self-emulsion. Klucel EF was added, and dissolved for about 20 minutes in the water.

The final emulsion was cast on a PET (Mylar™) web, using a tabletop casting machine with a knife space of 1000 μm. The cast emulsion was dried in an oven at 60° C. for about 60 minutes. The dried product was a film with a thickness of about 120 μm.

4 sheets of the dry film were stacked one on top of the other and laminated manually.

The laminated films have a thickness of about 500 μm.

### CR Layer:

An exemplary preparation of dispersion containing modified release agent and the preparation of a dry layer for CR (without drug), is presented below. The composition of a single unit is summarized in Table 7.

TABLE 7

| CR layer (without drug) | |
|---|---|
| formulation 1 | mg per unit |
| HPMC K100M | 75 |
| Klucel EF | 75 |
| PEG 400 | 30 |

In a 0.2-Liter planetary mixer, PEG 400+ Klucel EF were dissolved in ethanol, HPMC K100M was added, and dispersed for about 30 minutes in the ethanol. The final dispersion was cast on a PET (Mylar™) web, using a tabletop casting machine with a knife space of 1000 μm. The cast dispersion was dried in an oven at 50° C. for about 15 minutes. The dried product was a film with a thickness of about 200 μm.

### Lamination

The HPMC layer was laminated manually on the drug layer. The laminated film was punched into 21×45 mm rectangle units, each with weight of 700 mg, each unit comprising 10 mg CBD.

Each unit was rolled to cylinder shape, with the HPMC layer being the internal layer, and the drug layer being the outer layer.

The final unit was inserted into a 00 hard gelatin capsule.

The final units were tested to define the dissolution profile.

### Dissolution Profile:

The dissolution of the capsules containing the laminated Drug+CR layers were tested using the same method and parameters as described in Example 1.

The results of the dissolution test of Example 4 in SGF are presented in FIG. 4.

The release profile demonstrates prolonged and controlled release profile up to 10 hours.

### Example 5: CBD 20 mg CR Formulation 5-Prepared by HME (Hot Melt Extrusion)

### CR Fraction:

An exemplary preparation of an oral CR dosage form containing CBD micelles, is presented below. The composition of a single unit is summarized in Table 8 (same as Table 1).

TABLE 8

| CR formulation 1 | mg per unit | % In formulation |
|---|---|---|
| CBD | 20 | 3.2 |
| Peceol | 42 | 6.8 |
| Kolliphor EL | 98 | 15.8 |
| Klucel EF | 275 | 44.4 |
| Klucel GF | 135 | 21.8 |
| PEG 400 | 50 | 8.1 |
| Total | 620 | 100 |

CBD was dissolved in Peceol and Kolliphor mixture heated to 70° C. using magnetic stirrer to obtain a clear solution.

The solution was mixed manually with the other excipients prior to the hot melt extrusion process.

The final blend was inserted into a MC 15HT micro-compounder (Xplore) hot melt extrusion machine, and was processed under the following conditions:

Temperature: 180° C.

Rotation: 50 RPM.

Processing time: 5 minutes.

Screws configuration: counter rotation.

The final product is a uniform extrudate with diameter of 3 mm.

The extrudate was cut into about 7 cm length units, with weight of about 650 mg each comprising 20 mg CBD.

The unit were inserted into a 00 hard gelatin capsule.

The final units were tested to define the dissolution profile.

### Dissolution Profile:

The dissolution rates of the capsules were tested using the same method and parameters as described in Example 1.

The results of the dissolution test of Example 5 in SGF are presented in FIG. 5.

The release profile is linear profile demonstrating prolonged and controlled release profile up to 10 hours.

The final extrudate was inspected in scanning electron microscope according to the following procedure:

The SEM images were taken to the cross-section of the extrudate (gold coated) using Zeiss Ultra-Plus system with acceleration voltage of 4.0 kV. Images were taken using secondary emitted electrons (SE) detector. The SEM pictures of the extrudate are presented in FIG. 5A. The SEM pictures confirm that CBD was in emulsion form in the final extrudate.

### Example 6: THC 15 mg CR

An exemplary preparation of an emulsion containing THC and the preparation of an oral CR dosage form containing THC micelles, is presented below. The composition of a single unit is summarized in Table 9.

TABLE 9

| CR formulation 4 | mg per unit |
|---|---|
| THC | 15 |
| Sesame oil | 15 |
| Labrasol | 135 |
| Klucel EF | 250 |
| CMC 7MF | 75 |
| PEG 400 | 40 |

THC is dissolved in Labrasol and sesame oil mixture heated to 70° C. using magnetic stirrer to obtain a clear solution.

In a 0.2-Liter planetary mixer, PEG 400 is dissolved in water. The THC clear solution is added to form a self-emulsion. Klucel EF and CMC are added, and dissolved for about 60 minutes in the water. The final emulsion is cast on a PET (Mylar™) web, using a tabletop casting machine with a knife space of 1000 μm. The cast emulsion is dried in an oven at 60° C. for about 60 minutes. The dried product is a film with a thickness of about 150 μm.

5 sheets of the dry film are stacked one on top of the other and laminated manually.

The laminated films have a thickness of about 750 μm. The laminated film is punched into 21×45 mm rectangle units, each with weight of 600-650 mg, each unit comprising 15 mg THC.

The units are rolled to cylinder shape and are each inserted into a 00 hard gelatin capsule.

### Example 7: Botanical Cannabis Extract 30 mg CR

An exemplary preparation of an emulsion containing botanical cannabis extract and the preparation of an oral CR dosage form containing botanical cannabis extract micelles, is presented below. The composition of a single unit is summarized in Table 10.

TABLE 10

| CR formulation 5 | mg per unit |
|---|---|
| botanical cannabis extract | 30 |
| Labrafil ® M 2125 CS | 100 |
| Labrafac ™ lipophile WL 1349 | 20 |
| HPMC E3 | 200 |
| HPMC K4M | 80 |
| PEG 400 | 60 |

The cannabis extract is dissolved in Labrafac and Labrafil mixture heated to 70° C. using magnetic stirrer to obtain a clear solution.

In a 0.2-Liter planetary mixer, PEG 400 is dissolved in water, The cannabis extract clear solution is added to form a self-emulsion. HPMC E3 and K4M are added, and dissolved for about 60 minutes in the water.

The final emulsion is cast on a PET (Mylar™) web, using a tabletop casting machine with a knife space of 1000 μm. The cast emulsion is dried in an oven at 60° C. for about 60 minutes. The dried product is a film with a thickness of about 150 μm. 5 sheets of the dry film are stacked one on top of the other and laminated manually.

The laminated films have a thickness of about 750 μm. The laminated film is punched into 21×45 mm rectangle units, each with weight of 500-550 mg.

The units are rolled to cylinder shape and each is inserted into a 00 hard gelatin capsule.

### Example 8: Botanical Cannabis Extract 300 mg CR

An exemplary preparation of an emulsion containing botanical cannabis extract and the preparation of an oral CR dosage form containing botanical cannabis extract micelles, is presented below. The composition of a single unit is summarized in Table 11.

TABLE 11

| CR formulation 6 | mg per unit |
|---|---|
| botanical cannabis extract | 300 |
| GELUCIRE 48/16 | 200 |
| Klucel EF | 180 |
| Methyl cellulose A4M | 80 |
| PROPYLENE GLYCOL | 70 |

Botanical cannabis extract is dissolved in Gelucire 48/16 heated to 70° C. using magnetic stirrer to obtain a clear solution.

In a 0.2-Liter planetary mixer, PROPYLENE GLYCOL is dissolved in water. The cannabis clear solution is added to form a self-emulsion. Klucel EF and methyl cellulose A4M are added, and dissolved for about 60 minutes in the water.

The final emulsion is cast on a PET (Mylar™) web, using a tabletop casting machine with a knife space of 1100 μm. The cast emulsion is dried in an oven at 60° C. for about 60 minutes. The dried product is a film with a thickness of about 150 μm. 5 sheets of the dry film are stacked one on top of the other and laminated manually.

The laminated films have a thickness of about 750 μm. The laminated film is punched into 21×45 mm rectangle units, each with weight of 800-mg, each unit comprising 300 mg botanical cannabis extract.

The units are rolled to cylinder shape and each is inserted into a 00 hard gelatin capsule.

### Example 9-Ritonavir 100 mg CR

An exemplary preparation of an emulsion containing Ritonavir and the preparation of an oral CR dosage form containing Ritonavir micelles, is presented below. The composition of a single unit is summarized in Table 12.

TABLE 12

| CR Formulation 7 | mg per inner unit |
|---|---|
| Ritonavir | 100 |
| Labrasol | 60 |
| Kolliphor RH 40 | 60 |
| PVP K30 | 180 |
| Klucel GF | 150 |
| POLOXAMER 407 | 35 |

Ritonavir is dissolved in Labrasol and Kolliphor mixture using magnetic stirrer to achieve clear solution.

In a 0.2-Liter planetary mixer, POLOXAMER 407 is dissolved in water.

The Ritonavir clear solution is added to form self-emulsion, PVP K30 and Klucel GF are added and dissolved, for 90 minutes.

The final emulsion is cast on a silicon-coated PET (Mylar™) web, using a tabletop casting machine with a knife space of 1200 μm. The cast emulsion is dried in an oven at 60° C. for about 90 minutes. The dried product is a film with a solvent content (based on a loss-on-drying test) value of not more that 6%, and a thickness of about 150 μm.

5 sheets of the dry film are stacked one on top of the other and laminated manually.

The laminated films have a thickness of about 750 μm.

The laminated film is punched into 21×45 mm rectangle units, each with weight of 600-650 mg, each unit comprising 100 mg Ritonavir.

The units are rolled to cylinder shape and each is inserted into a 00 hard gelatin capsule.

### Example 10—Fenofibrate 50 mg CR

An exemplary preparation of an emulsion containing Fenofibrate and the preparation of an oral CR dosage form containing Fenofibrate micelles, is presented below. The composition of a single unit is summarized in Table 13.

TABLE 13

| CR Formulation 8 | mg per inner unit |
|---|---|
| Fenofibrate | 50 |
| Maisine ® CC | 30 |
| TWEEN 80 | 70 |
| CMC 7L2P | 230 |
| HEC HX | 70 |
| TEC | 50 |

Fenofibrate is dissolved in Maisine and Tween mixture using magnetic stirrer to obtain a clear solution.

In a 0.2-Liter planetary mixer, TEC is dissolved in water. The Fenofibrate clear solution was added to form self-emulsion, CMC 7L2P and HPMC K15M are added and dissolved, for 90 minutes. The final emulsion is cast on a silicon-coated PET (Mylar™) web, using a tabletop casting machine with a knife space of 1200 μm. The cast emulsion is dried in an oven at 60° C. for about 90 minutes. The dried product is a film with a solvent content (based on a loss-on-drying test) value of not more that 6%, and a thickness of about 150 μm.

5 sheets of the dry film are stacked one on top of the other and laminated manually. The laminated films have a thickness of about 750 μm. The laminated film is punched into 21×45 mm rectangle units, each with weight of 500-600 mg, each comprising 50 mg Fenofibrate. The units are rolled to cylinder shape and each is inserted into a 00 hard gelatin capsule.

### Comparative Example 11—(CBD+THC IR Film)

### Ir Film:

An exemplary preparation of an emulsion containing THC and CBD and the preparation of an IR film containing THC and CBD micelles, is presented below. The composition of a single unit is summarized in Table 14.

TABLE 14

| IR film formulation A | mg per inner unit |
|---|---|
| THC | 10 |
| CBD | 10 |
| Labrasol | 80 |
| KOLLIPHOR RH40 | 20 |
| HPMC E3 | 180 |
| Klucel GF | 12 |
| TEC | 15 |

THC and CBD were dissolved in Labrasol and Kolliphor mixture using magnetic stirrer to obtain a clear solution.

In a 0.2-Liter planetary mixer, TEC was dissolved in water.

The THC and CBD clear solution was added to form a self-emulsion. HPMC E3 and Klucel GF were added and dissolved, for 90 minutes. The final emulsion was cast on a silicon-coated PET (Mylar™) web, using a tabletop casting machine with a knife space of 1100 μm. The cast emulsion was dried in an oven at 60° C. for about 120 minutes. The dried product is a film with thickness of about 150 μm.

3 sheets of the dry film were stacked one on top of the other and laminated through a roller. The laminated films have a thickness of about 450 μm. The laminated film was punched into 18×40 mm rectangle units, each with weight of about 330 mg, comprising 10 mg THC and 10 mg CBD.

The final units were tested to define the dissolution rate using the same method and parameters as described in Example 1.

The units completely dissolved within 30-60 minutes.

### Example 12: CBD 15 mg CR TABLET (Modifying Release Agent into the Film)

An exemplary preparation of an emulsion containing CBD and the preparation of a CR tablet containing CBD micelles, is presented below. The composition of a single tablet is summarized in Table 15.

TABLE 15

| Tablet formulation 1 | mg per unit |
|---|---|
| CBD | 15 |
| Sesame oil | 15 |
| Labrasol | 85 |
| Klucel EF | 250 |
| CMC 7MF | 75 |
| PEG 400 | 30 |
| MCC | 40 |
| Lactose anhydrous | 60 |
| PVP K30 | 10 |
| Magnesium stearate | 5 |

CBD is dissolved in Labrasol and sesame oil mixture heated to 70° C. using magnetic stirrer to obtain a clear solution.

In a 0.2-Liter planetary mixer, PEG 400 is dissolved in water. The CBD clear solution is added to form a self-emulsion. Klucel EF and CMC are added, and dissolved for about 60 minutes in the water.

The final emulsion is cast on a PET (Mylar™) web, using a tabletop casting machine with a knife space of 1000 μm. The cast emulsion is dried in an oven at 60° C. for about 60 minutes. The dried product is a film with a thickness of about 150 μm.

The film is milled using Hammer Mill. The milled film is sieved with 500-micron sieve.

MCC, PVP and Lactose are added to the sieved powder and blended for about 15 minutes. Magnesium stearate is added and blended for about 2 minutes.

The final blend is pressed into 18*6 mm tablet using tablet press machine, each with weight of about 600 mg, each unit comprising 15 mg CBD.

### Example 13: THC 20 mg CR TABLET (Modifying Release Agent Added as Powder)

An exemplary preparation of an emulsion containing THC and the preparation of an CR tablet containing THC micelles, is presented below. The composition of a single tablet is summarized in Table 16.

TABLE 16

| Tablet formulation 2 | mg per unit |
|---|---|
| THC | 10 |
| Peceol | 21 |
| GELUCIRE 50/13 | 49 |
| Klucel EF | 250 |
| Klucel GXF | 80 |
| PEG 400 | 30 |
| Dicalcium phosphate dihydrate | 75 |
| Mannitol | 30 |
| Magnesium stearate | 5 |

THC is dissolved in Peceol and GELUCIRE mixture heated to 70° C. using magnetic stirrer to obtain a clear solution.

In a 0.2-Liter planetary mixer, PEG 400 is dissolved in water. The CBD clear solution is added to form a self-emulsion. Klucel EF is added, and dissolved for about 60 minutes in the water.

The final emulsion is cast on a PET (Mylar™) web, using a tabletop casting machine with a knife space of 1000 μm. The cast emulsion is dried in an oven at 60° C. for about 60 minutes. The dried product is a film with a thickness of about 150 μm.

The film is milled using Hammer Mill. The milled film is sieved with 500-micron sieve. Dicalcium phosphate dihydrate, Mannitol and Klucel GXF are added to the sieved powder and blended for about 15 minutes. Magnesium stearate is added and blended for about 2 minutes.

The final blend is pressed into 16*5 mm tablet using tablet press machine, each with weight of about 600 mg, each unit comprising 15 mg CBD.

### Example 14: CBD 20 mg CR TABLET (by Hot Melt Extrusion)

An exemplary preparation of an emulsion containing CBD and the preparation of an CR tablet containing CBD micelles, is presented below. The composition of a single tablet is summarized in Table 17.

TABLE 17

| Tablet formulation 3 | mg per unit |
|---|---|
| CBD | 20 |
| Peceol | 42 |
| Kolliphor EL | 98 |
| Klucel EF | 275 |
| Klucel GF | 135 |
| PEG 400 | 50 |
| MCC | 40 |
| Lactose anhydrous | 60 |
| PVP K30 | 10 |
| Magnesium stearate | 5 |

An extrudate containing CBD micelles is prepared as described in Example 5. The extrudate is milled using Hammer Mill. The milled film is sieved with 500 micron sieve. Dicalcium phosphate dihydrate, mannitol and Klucel GXF are added to the sieved powder and blended for about 15 minutes. Magnesium stearate is added and blended for about 2 minutes.

The final blend is pressed into 20*7 mm tablet using tablet press machine, each with weight of about 750 mg, each unit comprising 15 mg CBD.

### Example 15: Pharmacokinetic Clinical Study Testing CR Formulations Described in Example 1, 2 and 3

| | |
|---|---|
| Study Title | A Single-Dose, Randomized, 4-Way Crossover Study to Compare the |
| | Safety, Tolerability and Pharmacokinetics of Controlled Release CBD |
| | formulations with Immediate release CBD capsules in Healthy Adult |
| | Volunteers |
| Investigational | CBD 5 mg IR and 20 mg CR formulation 1 (Administration A) |
| Drugs | CBD 5 mg IR and 20 mg CR formulation 2 (Administration B) |
| | CBD 25 mg IR and 225 mg CR formulation 3 (Administration C) |
| Reference Product | CBD 25 mg IR (CBD in sesame oil) (Administration D) |
| Study Objectives | To investigate the pharmacokinetics profiles of 3 CR formulations, |
| | following single oral dose. |
| | To compare the pharmacokinetic profiles of CBD following a single |
| | administration CBD IR formulation |
| | To monitor the safety and tolerability in these subjects. |
| Criteria for | Pharmacokinetic parameters (Cmax, Tmax, AUC0-t, AUC0-inf, Tlag, |
| Evaluation | Kel, T½) determined from plasma concentrations of CBD. |
| Study Design and | Single-center, open-label, single-dose, randomized, 4-way crossover |
| Procedures | comparative bioavailability study in healthy adults. |
| | 18 subjects are to participate in the study. Each subject undergoes |
| | screening procedures within 28 days prior to first dosing, to assess his |
| | eligibility to participate in the study. |
| | Eligible subjects will participate in three dosing periods. They will be |
| | randomized to one of 4 administration sequences. In each period |
| | subjects will be admitted to the CRC on the evening before dosing. On |
| | the next morning, after having consumed a standardized breakfast |
| | within 30 minutes before dosing they will receive one of the following |
| | administrations, according to a randomization list: |
| | Administration A: CBD 5 mg IR and 20 mg CR formulation 1 |
| | Administration B: CBD 5 mg IR and 20 mg CR formulation 2 |
| | Administration C: CBD 25 mg IR and 225 mg CR formulation 3 |
| | Administration D: CBD 25 mg IR (CBD in sesame oil) |
| | Drug administration will be followed by PK blood sampling and AE |
| | monitoring for the next 24 hours, at time points specified below. |
| | There will be a washout period of at least 7 days between each dosing. |
| Number of Subjects | (18) subjects are planned to participate in this study. |
| PK Evaluation | Timing of PK blood sampling |
| | Blood samples to determine plasma concentrations of CBD will be |
| | collected at the following time points, in each drug administration: |
| | Pre-dose (within 90 min before dosing), 0.5, 1, 2, 3, 4, 5, 6, 8, 10, 12, |
| | 18 and 24 hours post dose (a total of 13 samples per dosing period) |
| | Calculated PK Parameters: |
| | The following PK parameters will be estimated for each subject's |
| | plasma CBD concentrations: Cmax, Tmax, AUC0-t, AUC0-inf, Kel, |
| | Tlag and T½. |
| | Additional PK parameters will be calculated if deemed necessary. |

### Example 16: Leuprorelin-Docusate Complex 10 mg CR

An exemplary preparation of an emulsion containing leuprorelin-docusate complex and the preparation of an oral CR dosage form containing leuprorelin-docusate micelles, is presented below. The composition of a single unit is summarized in Table 18.

TABLE 18

| CR Formulation 9 | mg per inner unit |
|---|---|
| leuprorelin | 10 |
| Sodium docusate | 5 |
| Kolliphor EL | 120 |
| HPMC E3 | 300 |
| Klucel GF | 150 |
| POLOXAMER 407 | 35 |

### Hydrophobic Ion Pairing (HIP) Process:

Peptide solution is prepared by dissolving leuprorelin in 0.1 N HCl at a concentration of 10 mg/ml. (Griesser, G., et al., Int. J. Pharmaceutics, 520(1-2), 267-274 (2017)).

Surfactant solution is prepared by dissolving sodium docusate in water at a concentration of 5 mg/ml. The surfactant solution is added to the peptide solution under vigorous stirring (400 rpm). During the reaction, an immediate white precipitation indicates the formation of the hydrophobic peptide complex. The mixture is centrifuged at 4000 rpm for 10 min with High-Speed Centrifuge. The supernatant is separated from the precipitate and the final precipitate contains the leuprorelin-docusate complex is dried in an oven at 40° C. for 24 hours.

### Final Preparation:

The leuprorelin-docusate complex is dissolved in Kolliphor EL using magnetic stirrer to achieve clear solution.

In a 0.2-Liter planetary mixer, POLOXAMER 407 is dissolved in water.

The leuprorelin-docusate complex clear solution is added to form self-emulsion, HPMC E3 and Klucel GF are added and dissolved, for 90 minutes.

The final emulsion is cast on a silicon-coated PET (Mylar™) web, using a tabletop casting machine with a knife space of 1000 μm. The cast emulsion is dried in an oven at 60° C. for about 90 minutes. The dried product is a film with a solvent content (based on a loss-on-drying test) value of not more that 6%, and a thickness of about 150 μm.

5 sheets of the dry film are stacked one on top of the other and laminated manually.

The laminated films have a thickness of about 750 μm.

The laminated film is punched into 21×45 mm rectangle units, each with weight of 600-650 mg, each unit comprising 15 mg leuprorelin-docusate complex (10 mg leuprorelin).

The units are rolled to cylinder shape and each is inserted into a 00 hard gelatin capsule.

1.-66. (canceled)

67. An oral drug delivery device for controlled delivery of at least one lipophilic drug, the device comprising a CR drug-containing polymeric component comprising at least one pharmaceutically acceptable film-forming hydrophilic polymeric component, at least one pharmaceutically acceptable release-modifying agent, optionally at least one pharmaceutically acceptable plasticizer, and at least one lipophilic drug emulsified in at least one emulsifying agent embedded in said polymeric component,
wherein the oral drug delivery device is configured for being integrated into or is formed as an orally administrable dosage unit form, and
wherein said oral drug delivery device is configured for enabling said at least one release-modifying agent, upon ingestion of said dosage unit form and contact with gastric medium and/or intestinal medium, to form a viscous gel that is configured for gradually eroding concurrently with passage through the stomach and the intestinal tract, said gel controllably releasing said emulsified drug into surrounding environment in the form of micelles.

68. An oral drug delivery device of claim 67, wherein said release-modifying agent is a hydrophilic polymer which when dissolved in water at 2% w/w gives a solution with viscosity of at least 150 mPa*sec, optionally wherein said release modifying agent is comprised in an amount of at least about 30 mg per said dosage unit form, optionally wherein said dosage unit form is of total weight of from about 250 to about 2000 mg, optionally wherein said at least one lipophilic drug has log P>2.

69. An oral drug delivery device of claim 67, wherein said at least one emulsified drug is a pharmaceutically active cannabinoid, or a mixture of at least two pharmaceutically active cannabinoids, or a cannabis extract, ritonavir, fenofibrate, diclofenac, flurbiprofen diazepam, vitamin E, piroxicam, lopinavir, clofazimine, or a modified lipophilic drug, or a mixture of at least two pharmaceutically active cannabinoids, or a cannabis extract.

70. An oral drug delivery device of claim 69, comprising from about 0.5 to about 500 mg of said lipophilic drug per dosage unit form.

71. An oral drug delivery device of claim 67, wherein said dosage unit form comprises said drug delivery device integrated into a peroral capsule, optionally wherein said dosage unit form further comprises an immediate release IR (immediate release) drug-containing component, which IR component comprises an emulsion of said at least one lipophilic drug in at least one emulsifying agent, said IR drug-containing component optionally further comprising a suitable carrier or diluent wherein said IR drug-containing component is in solid or liquid form, optionally wherein said capsule is coated with said IR component.

72. An oral drug delivery device of claim 67, wherein said delivery device comprises from 1 to about 50 CR drug-containing layers, said CR drug-containing layers being laminated, each said CR drug-containing layer comprising said at least one emulsified lipophilic drug embedded in said polymeric component, each layer being of essentially rectangular, square or other polygonal shape or non-polygonal shape, optionally wherein the thickness of each said drug-containing layer is from about 20 μm to about 1000 μm, wherein said layers each has identical or different thickness, optionally wherein said laminated drug-containing layers are rolled together into an essentially cylindrical body.

73. An oral drug delivery device of claim 72, wherein said drug-containing member is integrated into a peroral capsule, wherein said capsule optionally further comprises or is coated with an immediate release (IR) drug-containing component, which comprises an emulsion of said at least one lipophilic drug in at least one emulsifying agent, said IR drug-containing component optionally further comprising a suitable carrier or diluent wherein said IR drug-containing component is in solid or liquid form.

74. An oral drug delivery device of claim 67, wherein said at least one pharmaceutically acceptable film-forming hydrophilic polymeric component, said at least one pharmaceutically acceptable release-modifying agent, said at least one optional pharmaceutically acceptable plasticizer, and said at least one lipophilic drug emulsified in said at least one emulsifying agent embedded therein constitute an essentially homogenous essentially solid CR mixture, configured for being integrated into said orally administrable dosage unit form, optionally wherein said CR mixture is essentially shaped as a cylinder of a diameter of from about 0.2 cm to about 1 cm, optionally wherein said dosage unit form is a peroral capsule, optionally wherein said capsule optionally further comprises and/or is coated with an immediate release (IR) drug-containing component, which comprises an emulsion of said at least one lipophilic drug in at least one emulsifying agent, said IR drug-containing component optionally further comprising a suitable carrier or diluent wherein said IR drug-containing component is in solid or liquid form.

75. An oral drug delivery device of claim 67, wherein said drug delivery device is configured as a CR orally administrable tablet, said tablet comprising a first said film-forming polymer and said optional plasticizer and said at least one lipophilic drug emulsified in at least one emulsifying agent embedded therein, and a release modifying agent optionally together with a further film-forming polymer which may be the same or different from said first film-forming polymer, wherein said tablet optionally further comprises at least one tableting excipient or additive, wherein said excipient or additive is any one of binder, diluent, filler, lubricant and glidant, and any mixture of at least two thereof, optionally wherein said tablet further comprises a further amount of at least one additional pharmaceutically acceptable release-modifying polymer in solid form, wherein said additional release modifying may be the same or different from said at least one, respectively, additional, pharmaceutically acceptable release-modifying agent, optionally wherein said tablet is optionally coated with an IR drug-containing component, which comprises an emulsion of said at least one lipophilic drug in at least one emulsifying agent, said IR drug-containing component optionally further comprising a suitable carrier or diluent wherein said IR drug-containing component is in solid or liquid form.

76. An oral drug delivery device of claim 75, wherein said tablet is contained in a peroral capsule, wherein said capsule optionally further comprises an immediate release (IR) drug-containing component, which comprises an emulsion of said at least one lipophilic drug in at least one emulsifying agent, said IR drug-containing component optionally further comprising a suitable carrier or diluent wherein said IR drug-containing component is in solid or liquid form.

77. An oral drug delivery device for controlled delivery of at least one lipophilic drug, the device comprising
a drug-containing polymeric component comprising at least one pharmaceutically acceptable film-forming hydrophilic polymeric component, optionally at least one pharmaceutically acceptable plasticizer, and at least one lipophilic drug emulsified in at least one emulsifying agent, wherein said emulsified drug is embedded in said drug-containing polymeric component,
a controlled release (CR) polymeric component comprising at least one pharmaceutically acceptable film-forming hydrophilic polymer, at least one pharmaceutically acceptable release-modifying agent, and optionally at least one pharmaceutically acceptable plasticizer,
wherein said hydrophilic film-forming polymer and said optional plasticizer in said drug-containing polymeric component and in said CR polymeric component may be the same or different; and
wherein the oral drug delivery device is configured for being integrated into an orally administrable dosage unit form, and
wherein said oral drug delivery device is configured for enabling said at least one release-modifying agent, upon ingestion of said dosage unit form and contact with gastric medium and/or intestinal medium, to form a viscous gel that is configured for gradually eroding concurrently with passage through the stomach and the intestinal tract, and for controllably releasing said emulsified drug into surrounding environment in the form of micelles, optionally wherein said release-modifying agent is a hydrophilic polymer which when dissolved in water at 2% w/w gives a solution with viscosity of at least 150 mPa*sec,
optionally, wherein said release modifying agent is comprised in an amount of at least about 30 mg per said dosage unit form and wherein said at least one emulsifying agent is a lipid excipient having HLB (Hydrophilic Lipophilic Balance) greater than 6, optionally wherein said emulsifying agent is a mixture of lipid excipients of which at least 50% have HLB greater than 6, and remaining lipid excipients are any one of oils, fatty acids, glycerides, polyoxyglycerides, polyglyceryl and polyalcohol esters or water insoluble surfactants, or any mixture of at least two thereof, wherein each said remaining lipid excipients has HLB lower than, equal to or higher than 6.

78. An oral drug delivery device of claim 77, wherein said drug-containing polymeric component comprises from 1 to about 50 drug-containing layers, each said layer comprising said at least one emulsified lipophilic drug embedded in said hydrophilic polymeric component and optionally said plasticizer, said drug-containing layers being laminated, each said drug-containing layer being of essentially rectangular, square or other polygonal shape or non-polygonal shape, optionally wherein the thickness of each said drug-containing layers is from about 15 μm to about 900 μm, wherein said layers each has identical or different thickness optionally wherein said drug-containing polymeric component and said CR polymeric component are laminated together, optionally wherein said laminated drug-containing polymeric component and CR polymeric component are rolled together forming an essentially cylindrical body, optionally wherein said drug delivery device is integrated into a peroral capsule, wherein said capsule optionally further comprises and/or is coated with an immediate release (IR) drug-containing component, which comprises an emulsion of said at least one lipophilic drug in at least one emulsifying agent, said IR drug-containing component optionally further comprising a suitable carrier or diluent wherein said IR drug-containing component is in solid or liquid form.

79. An oral drug delivery device of claim 78 wherein said dosage unit form is of total weight of from about 250 to about 2000 mg, optionally comprising said at least one emulsified lipophilic drug at from about 1 to about 65% w/w.

80. An oral drug delivery device of claim 69, wherein said pharmaceutically active cannabinoid is any one of Δ-9-tetrahydrocannabinol (Δ9-THC, THC), iso-tetrahydrocannabimol (iso-THC), cannabinol (CBN), cannabidiol (CBD) cannabigerol (CBG), cannabichromene (CBC), cannabielsoin (CBE), cannabicyclol (CBL), cannabicitran (CBT), cannabivarin (CBV), tetrahydro-cannabivarin (THCV), cannabidivarin (CBDV), tetrahydrocannabidiol (THCBD), tetrahydrocannabigerol (THCBG), tetrahydrocannabichromene (THCBC), tetrahydrocannabidivarol (THCBDV), cannabichromevarin (CBCV), cannabigerovarin (CBGV) and cannabigerol conomethyl ether (CBGM) and pharmaceutically acceptable derivatives and pharmaceutically active metabolites thereof.

81. An oral drug delivery device of claim 78, wherein said at least one release-modifying agent is any one of methylcellulose, hydroxypropyl methylcellulose, hydroxypropyl cellulose, polyethylene oxide, carboxymethyl cellulose, gelatin, a gum and a protein, and any mixture of at least two thereof.

82. An oral drug delivery device of claim 78, wherein said lipid excipient with HLB value above 6 is any one of Gelucire 50/13 (stearoyl polyoxyl-32 glycerides), Gelucire 44/14 (lauroyl polyoxyl-32 glycerides), Gelucire 48/16 (polyoxyl-32 stearate (type I) NF), Labrafil 2125 (linoleoyl polyoxyl-6 glycerides), Labrafil 1944 (oleoyl polyoxyl-6 glycerides), Labrasol (caprylocaproyl polyoxyl-8 glycerides), Kolliphor RH40 (polyoxyl 40 hydrogenated castor oil), Kolliphor EL (polyoxyl-35 castor oil), Kolliphor HS 15 (macrogol-15-hydroxystearate), Kolliphor P 407/124/188 (poloxamer 407/124/188) and Kolliphor PS 80/60/20 (polysorbate 80/60/20), and any mixture of at least two thereof, optionally wherein remaining lipid excipients are any one of oils, fatty acids, glycerides, remaining lipid excipients are any one of oils, fatty acids, glycerides, optionally wherein the weight ratio lipophilic drug:emulsifying agent is from about 2:1 to about 1:100.

83. An oral drug delivery device of claim 78, wherein said hydrophilic film-forming polymer is any one of povidone, copovidone, polyvinyl alcohol, hydrophilic polyacrylamide derivatives, proteins, gelatin, hydroxypropyl cellulose, polyethylene oxide, amino-methacrylate copolymer NF, hydroxypropyl methylcellulose, hydroxyethyl cellulose, carboxymethyl cellulose, sodium carboxymethylcellulose, methylcellulose, or polyvinyl alcohol-polyethylene glycol graft copolymer and any combination of at least two thereof, optionally wherein the film-forming polymer and the release-modifying agent together constitute from about 25 to about 85% w/w, wherein said drug delivery device optionally comprises said plasticizer at up to 20% w/w.

84. An orally administrable pharmaceutical dosage unit form comprising a drug delivery device as defined in claim 78, configured for being integrated into a peroral capsule, wherein the delivery device comprises as said active ingredient at least one pharmaceutically active cannabinoid or mixture of at least two pharmaceutically active cannabinoids or cannabis extract.

85. A method for any one of treating, alleviating and preventing worsening of a disease, disorder or condition responsive to cannabinoid therapy in a subject in need, said method comprising orally administering to said subject an oral drug delivery device as defined in claim 77, wherein said administration is under fed or under fasted conditions, optionally wherein said disease, disorder or condition responsive to cannabinoid therapy is any one of anorexia associated with weight loss in patients with AIDS, nausea and vomiting associated with cancer chemotherapy, pain, anxiety, depression, muscle spasticity, arthritis and rheumatism, multiple sclerosis and other neuromuscular inflammatory disorders, inflammatory bowel diseases, Crohn's disease, colitis, post-traumatic stress disorder (PTSD), epileptic seizures, Parkinson's disease, spinal cord injury, fibromyalgia, Alzheimer's disease and dementia or any other condition responsive to cannabinoid therapy.

86. A method for any one of treating, alleviating and preventing worsening of a disease, disorder or condition responsive to any one of ritonavir, fenofibrate, diclofenac, flurbiprofen diazepam, vitamin E, piroxicam, lopinavir, clofazimine, or a modified lipophilic drug, said method comprising orally administering to said subject an oral drug delivery device as defined in claim 78, wherein said administration is under fed or under fasted conditions.
